(19) United States
(12) Patent Application Publication
(10) Pub. No.: US 2019/0167569 A1
(43) Pub. Date: Jun. 6, 2019

(54) ENHANCED HERBAL EXTRACTION AND TRANSDERMAL DELIVERY

(71) Applicant: Nuri Murat ÖZAYMAN, Bahçelievler/Istanbul (TR)

(72) Inventor: Nuri Murat ÖZAYMAN, Bahçelievler/Istanbul (TR)

(21) Appl. No.: 16/314,602

(22) PCT Filed: Jun. 30, 2017
(86) PCT No.: PCT/TR2017/050293

§ 371 Date: Dec. 31, 2018

(30) Foreign Application Priority Data
Jul. 1, 2016 (TR) 2016/09387

(51) Int. Cl. A61K 9/00; A61K 9/70; A61K 9/107; A61Q 17/00; A61Q 19/00

(52) U.S. Cl. CPC A61K 9/0014; A61K 9/7023; A61Q 19/00; A61Q 17/005; A61K 9/107

(57) ABSTRACT

Shortcomings in the transdermal delivery of an herbal extraction are overcome by using an aqueous extraction of a saponin plant to enhance herbal extraction from a subject plant and to enhance transdermal delivery of a composition including the herbal extraction. In one embodiment, a composition for transdermal delivery comprises an herbal extract solution having soluble components of a subject plant solvated in an extraction medium. The extraction medium includes soluble components of a saponin plant solvated in water, and the saponin plant and the subject plant are different plants or types of plants. Products including the composition for transdermal delivery, and methods of preparing and using the composition are also described.

### TECHNICAL FIELD

This invention relates to enhanced transdermal delivery of compositions comprising hydrophilic and/or lipophilic ingredients of a plant and manufacturing methods thereof.

### BACKGROUND

Public concern about the safety of synthetic, man-made ingredients or components used in personal care products, especially regarding their accumulation and subsequent heath effects, have driven health authorities to reduce the applied concentrations or even ban synthetic ingredients. Alternatives, such as plant substances, have become the focus of much research. Various consumer products, such as cosmetic products, soaps, cleaning products, other personal care products, and medical devices (hereafter also referred to as “products”), include more and more herbs as the demand from the conscious consumer increases for “natural” products that meet desired requirements.

Hydrophilic (i.e., water soluble) herbal or plant ingredients can be extracted by chemicals or water, and by various techniques, such as infusion, decoction, and maceration. A resultant liquid including extracted herbal or plant components is hereinafter referred to also as an “extract solution”.

Lipophilic (i.e., oil soluble) herbal or plant ingredients can be obtained either by pressing the necessary parts, mostly seeds, of the plant to obtain a fixed oil or by distilling out the essential oils of the desired parts, mostly leaves or flowers. An emulsion/dispersion of a fixed and/or essential oil in water (i.e., a fixed oil, an essential oil, or a combination of both) is hereinafter referred to also as an “oil dispersion”.

Infusion is the process of extracting chemical compounds or flavors from plant material in a solvent such as oil or alcohol, by allowing the material to remain suspended in the solvent over time (a process often called steeping). An infusion is also the name for the resultant liquid. In one example, an infusion includes the use of simmering water, which is poured over chosen subject or target plants or herbs. The brew is covered for a few minutes to produce an infusion or an extract solution by infusion. In a further example, the heat will release essential oils which are valuable for their concentrated active principles.

Decoction is a method of extraction by boiling of dissolved chemicals from herbal or plant material, which may include stems, roots, bark and rhizomes. Decoction may include first mashing and then boiling the plant in water to extract the plant components or other chemical substances. Decoctions and infusions may produce liquids with differing chemical properties as there are temperature/preparation differences. In one example, a decoction includes putting the plant material in cold water and bringing the mixture to boil. Once the water comes to a boil, the mixture is removed from the heat and covered for a few minutes to produce a decoction or an extract solution by decoction.

Maceration involves extraction by solvent extraction. In one example, a maceration includes placing the plant material in a container of cold water for 24 hours or longer to steep and extracting the active principles to thereby produce a maceration or an extract solution by maceration.

However, several problems have been encountered during herbal extraction processes, as described below.
1) Chemical solvents that are used for an extraction process may be undesired in an end product but remain in the extract and cause contamination even in small amounts. Solvents, such as carbon dioxide, may react with the basic components due to their acidic nature and remain in the extract as more stable compounds but nonetheless cause contamination. This contamination may cause complications for using the extract and in situations where the herbal remnants are desired for subsequent use, such as for animal feed.
2) Organic extracts that are stored for long periods of time have a higher risk of deterioration and becoming “stale”. Extracts that are produced in necessary amounts in the production area and then used are more assured to remain fresh and potent.
3) Extraction costs are increased by the need to devote capital funds for the foundation of facilities or equipment for the means of extraction, such as for super critical carbon dioxide use, and operational costs such as for separation of the desired extract from the solvent.

Accordingly, there is a need for methods that provide for desired natural extracts, and which eliminate or reduce to acceptable levels the shortcomings of prior herbal extraction means and methods as noted above.

Furthermore, oils, such as essential oils and/or fixed oils, have desirable characteristics for products such as perfumes, cosmetics, soaps, household cleaning products, medical devices, and other like products. Essential oils are also known as volatile oils, ethereal oils, aetherolea, or simply as the oil of the plant from which they were extracted. An oil is “essential” in the sense that it contains the “essence of” the plant's fragrance—the characteristic fragrance of the plant from which it is derived. Fixed oils are natural animal or vegetable oils that are not volatile and are also known as natural nonvolatile oils or fatty oils. Oils are typically hydrophobic or not miscible in water. Direct application of most essential oils and some fixed oils are irritating to the skin, and therefore they are either diluted in a better tolerated carrier oil or dispersed in water with the help of a dispersing agent/emulsifier. Prior typical dispersing or emulsifying agents have been synthetic in nature, and thus a natural (even edible) dispersing agent/emulsifier with advantageous function and results is highly desirable.

Many plant ingredients, including water soluble components or oils, may be topically applied with a purpose to pass through the skin. However, products including oils can often be difficult to deliver transdermally and may instead be easily washed away after remaining on the skin surface after application.

Thus, a solution using natural ingredients for enhancing both herbal extraction and transdermal delivery and/or enhancing both oil dispersion and transdermal delivery into deeper layers of skin is highly desirable for an efficient and effective composition and method of preparing certain consumer products for topical applications.

### SUMMARY

The present invention advantageously and surprisingly eliminates or reduces the above-mentioned shortcomings of prior herbal extraction and transdermal delivery means and methods by using an extraction medium including saponin for enhancing both herbal extraction and transdermal delivery. In particular, an aqueous extraction of a saponin plant is used to both enhance herbal extraction from a subject plant and enhance transdermal delivery of a composition including the herbal extraction of the subject plant.

Furthermore, the present invention advantageously and surprisingly eliminates or reduces the above-mentioned shortcomings of prior oil dispersion/emulsion and transdermal delivery means and methods. In particular, an aqueous extract of a saponin-containing plant is used as a dispersion medium to both enhance oil dispersion and enhance transdermal delivery of a composition including the oil dispersion. Thus, the dispersion medium of the present invention provides for a non-synthetic emulsifying agent or dispersing medium for oil, which also enhances transdermal delivery of the product including the oil. The dispersion medium of the present invention is formed of natural reactants and may even be edible in one embodiment.

As used herein, the term “extract” refers to an active ingredient or fraction isolated from a plant by using a solvent or a solvent system. Also as used herein, the term “extract solution” refers to the solution of the extract solvated in the solvent.

The extraction procedure for obtaining any of the plant extracts employed in accordance with the invention, unless otherwise indicated, may be carried out in various ways. For example, an herbal extract solution can be one of an infusion, a decoction, a maceration, or a product from another extraction technique performed on a subject plant by man. In yet another example, the plant parts can be crushed and/or milled and optionally dried before being contacted with the extraction solvent; the extraction can be assisted with shaking, agitating, and/or heating; the extraction can be microwave and/or ultrasound assisted; the solvent can be filtered and reduced under reduced pressure evaporation; the filtered solids may be re-extracted to yield a second crop; and so forth. In the alternatives set forth herein, a water extract of a saponin containing plant is employed as a solvent.

Herbal Extraction and Transdermal Delivery

In accordance with one embodiment, a composition for transdermal delivery comprises an herbal extract solution having soluble components of a subject plant solvated in an extraction medium. The extraction medium includes soluble components of a saponin plant solvated in water, and the saponin plant and the subject plant are different plants or types of plants. A subject plant may be one or more of various plants solvated in the extraction medium, and a saponin plant may be one or more of various plants solvated in a solvent, such as water.

In accordance with another embodiment, a composition for transdermal delivery comprises: an oily phase including an oil between 0.1 wt % and 6 wt % of the composition; an aqueous phase including an herbal extract solution having soluble components of a subject plant solvated in an extraction medium; and a preservative providing antioxidant and/or antimicrobial properties, wherein the preservative is between 0.1 wt % and 3 wt % of the composition. The herbal extract solution is one of an infusion, a decoction, a maceration, or a product from another extraction technique on the subject plant. The herbal extract solution is produced from an extraction technique on the subject plant using 1 weight unit of the subject plant to 6-12 weight units of the extraction medium. The extraction medium includes soluble components of a saponin plant solvated in water. The extraction medium is produced from an extraction technique on the saponin plant using 1 weight unit of the saponin plant to 5-10 weight units of water. The extraction medium is one of an infusion, a decoction, a maceration, or a product from another extraction technique on the saponin plant. The saponin plant and the subject plant are different plants or types of plants.

In yet another embodiment, a product is provided comprising any of a composition as described in the various embodiments above. The product may be selected from the group consisting of a cosmetic, a preservative formulation, an antimicrobial formulation, a pharmaceutical composition, a medical device, and a disinfectant.

In accordance with yet another embodiment, a method for treating human skin comprises applying, to a skin surface, a suitable composition as described above.

In accordance with yet another embodiment, a method for enhanced herbal extraction and transdermal delivery of an herbal extract solution comprises: extracting soluble components of a saponin plant with water to provide an extraction medium; extracting soluble components of a subject plant with the extraction medium to provide an herbal extract solution; and mixing the herbal extract solution with a component of complimentary function to produce a product for transdermal delivery.

Oil Dispersion and Transdermal Delivery

In accordance with one embodiment, a composition for transdermal delivery comprises an oil in water emulsion having an oil of a target plant dispersed in a dispersion medium, wherein the dispersion medium includes soluble components of a saponin plant solvated in water, and wherein the saponin plant and the target plant are different plants or types of plants. A target plant may be one or more of various plants from which the oil (fixed and/or essential) is provided, and a saponin plant may be one or more of various plants solvated in a solvent, such as water.

In accordance with another embodiment, a composition for transdermal delivery comprises: an oil in water emulsion having a fixed oil and/or an essential oil of a target plant dispersed in a dispersion medium; a preservative providing antioxidant and/or antimicrobial properties, wherein the preservative is between 0.1 wt % and 3 wt % of the composition; and a film-forming biopolymer, wherein the biopolymer is between 0.1 wt % and 4 wt % of the composition. The fixed oil and/or the essential oil of the target plant is between 0.1 wt % and 6 wt % of the composition. The dispersion medium includes soluble components of a saponin plant solvated in water, the dispersion medium is produced from an extraction technique on the saponin plant using 1 weight unit of the saponin plant to 5-10 weight units of water, and the dispersion medium is one of an infusion, a decoction, a maceration, or a product from another extraction technique on the saponin plant. The saponin plant and the target plant are different plants or types of plants.

In accordance with yet another embodiment, a product comprises any of a composition as described above, wherein the product is selected from the group consisting of a cosmetic, a preservative formulation, an antimicrobial formulation, a pharmaceutical composition, a medical device, and a disinfectant.

In accordance with yet another embodiment, a method for treating human skin comprises applying, to a skin surface, a suitable composition as described above.

In accordance with yet another embodiment, a method for enhanced oil dispersion and transdermal delivery of an oil comprises: extracting soluble components of a saponin plant with water to provide a dispersion medium; dispersing an oil in the dispersion medium to provide an emulsion; and mixing the emulsion with a component of complimentary function to produce a product for transdermal delivery.

### DETAILED DESCRIPTION

The present invention advantageously and surprisingly eliminates or reduces the above-mentioned shortcomings of prior herbal extraction and transdermal delivery means and methods by using an extraction medium including saponin for enhancing both herbal extraction and transdermal delivery. In particular, an aqueous extraction of a saponin plant is used to both enhance herbal extraction from a subject plant and enhance transdermal delivery of a composition including the herbal extraction of the subject plant.

Furthermore, the dispersion medium of the present invention advantageously provides for a non-synthetic emulsifying agent or dispersing medium for oil, which also enhances transdermal delivery of the product including oil. The dispersion medium of the present invention is formed of natural reactants and may even be edible. In one embodiment, an essential oil and/or a fixed oil is dispersed in water and also rendered transdermal with a dispersion medium including a saponin content.

Compositions for Transdermal Delivery (Topical Application)

I. Herbal Extract Compositions

In accordance with one embodiment, a composition for transdermal delivery comprises an herbal extract solution having soluble components of a subject plant solvated in an extraction medium. The extraction medium includes soluble components of a saponin plant solvated in water, and the saponin plant and the subject plant are different types of plants.

The above composition may have the following alternative components, which may also be combined in various applicable and functioning combinations within the scope of the present invention.

The subject plant may be selected from the group consisting of: Achillea Millefolium, Aesculus Hippocastanum, Agrimonia Aupatoria, Alchemilla Vulgaris, Allium Sativum, Althaea Officinalis, Angelica Archangelica, Angelica sinensis syn. A. polymorpha, Anthemis Nobilis, Apium Graveolens, Arctostaphylos Uva Ursi, Arnica Montana, Artemisia Vulgaris, Articum Lappa, Aspalathus Linearis, Astragalus membranaceus, Berberis Vulgaris, Betula Alba, Borago Officinalis, Calendula Officincalis, Calluna Vulgaris, Camellia Sinensis, Capsella Bursa Pastoris, Capsicum Annuum, Cardus Benedictus, Cardus Marianus, Carum Carvi, Cassia Angustifolia, Centella asiatica syn. Hydrocotyle, Ceranus Vulgaris, Ceratonia siliqua, Chamaecrista mimosoides (L.), Cichorium Intybus, Cimicifuga racemosa, Cinnamomum Zeylanicum, Citrus paradisi Macfadyen, Citrus Aurantium, Citrus Limonum, Citrus Sinensis, Cnidium monnieri, Coffea Cruda, Cordyceps sinensis (Berk.) Sacc., Cortex Magnoliae Officinalis, Corylus Avellana, Crataegus monogyna, Crataegus cuneata Sieb. et Zucc., Crataegus Oxyacantha, Cucurbita Pepo, Cupressus Sempervirens, Curcuma Longa, Cymbopogon Flexuosus, Cyminum Cuminum, Cynara Scolymus, Daucus Carota, Dioscoreavillosa, Echinacea Angustifolia, Echinacea Purpurea, Elettaria Cardamomum, Eleutherococcus Senticosus, Eleutherococcus senticosus, Acanthopanax senticosus, Epimedium brevicornum, Equisetum Arvense, Erigeron Canadensis, Eschscholtzia Califomica, Eucalyptus Globulus, Ferula elaeochytris, Foeniculum Vulgare, Fragaria Vesca, Fraxinus Exelsior, Fructus Schisandrae, Fumaria Officinalis, Galla Chinensis, Gentiana Lutea, Ginkgo Biloba, Glechoma Hederaceum, Glycine max, Glycyrrhiza Glabra, Hamamelis Virginiana, Harpagophytum, Hibiscus Sabdariffa, Hieracium Pilosella, Humulus Lupulus, Hypericum Perforatum, Hysopus Officinalis, Juglans Regia, Juniperus Communis, Lavandula Angustifolia, Levisticum Officinalis, Ligustrum lucidum, Ait. or Eriobotrya japonica (Thunb.) Lindl. L., Lippia Citriodora, Lotus Comiculatus, Lycium Barbarum, Lycopersicum esculentum, Malva Sylvestris, Matricaria Chamomilla, Medicago Sativa, Melilotus Officinalis, Melissa Officinalis, Mentha Piperita, Mentha Viridis, Momordica Charantia, Ocimum Basilicium, Olea Europea, Ononis Spinosa, Origanum Majorana, Origanum Vulgaris, Paeonia Albiflora Root, Paeonia suffruticosa, Passiflora Incarnata, Passiflora quadrangularis, Paullinia Cupana, Perilla frutescens (L.) Britton., Phaseolus Vulgaris, Pimpinella Anisum, Pinus Pinaster, Pinus strobes, Pinus Sylvestris, Plantago Lanceolata, Plantago Major, Pneumus Boldus, Polygonum cuspidatum sieb. et zucc, Primula Officinalis, Pueraria lobata (Willd.) Ohwi, Pulmonaria Officinalis, Punica granatum L., Punica Granatum, Pyrus malus, Quercus Robur, Quercus spp., Raphanus Sativus, Rhamnus Frangula, Rheum Officinalis, Rhodiola rosea L., Ribers Nigrum, Rosa Canina, Rosmarinus Officinalis, Rubus fructicosus, Rubus ldeaus, Ruscus Aculeatus, Sabal Serrulata, Salix Alba, Salvia miltiorrhiza, Salvia Officinalis, Salvia Sclarea, Sambucus Nigra, Satureja Montana, Serenoa repens, Silybum marianum (L.) Gaertner, Soja Hispida, Solidago Virga Aurea, Spinacia Oleracea, Spirea Ulmaria, Symphytum officinalis, Taraxacum officinale, Taraxacum Officinalis, Thymus sp., Thymus vulgaris, Tilia Officinalis, Tribulus terrestris, Trigonella, Uncaria Tomentosa, Urtica Dioica, Valeriana Officinalis, Verbascum Thapsus, Verbena Officinalis, Viburnum Lanta, Viola Tricolor, Viscum Album, Vitex Agnus Castus, Vitis Vinifera, Zea Mais, Zingiber Officinalis, and a combination thereof. This group may be referred to as a subject plant list.

The herbal extract solution may be one of an infusion, a decoction, a maceration, or a product from another extraction technique performed on the subject plant by man.

The saponin plant may be selected from the group consisting of: Camellia sinensis, Styrax japonica, Acacia concinna, Acacia nilotica, Acorus calamus, Aesculus hippocastanum, Agave Americana, Ailanthus altissima, Akebia quinata, Albizia julibrissin, Aletris farinose, Aleurites fordii, Allium cepa, Allium drummondii, Allium fistulosum, Allium neapolitanum, Allium oleraceum, Allium ramosum, Allium sativum var. sativum, Allium schoenoprasum, Allium sphaerocephalon, Allium tricoccum, Allium tuberosum, Allium ursinum, Allium vineale, Anemone pulsatilla, Arisaema consanguineum, Asparagus lucidus, Balanites aegyptiacus, Banisteriopsis caapi, Bupleurum falcatum, Calendula officinalis, Camellia oleifera, Capparis spinosa, Capsicum annuum, Castanospermum austral, Caulophyllum thalictroides, Centella asiatica, Ceratonia siliqua, Chrysophyllum cainito, Cissampelos pareira, Coix lacryma-jobi, Colocasia esculenta, Colubrina arborescens, Corchorus olitorius, Cymbopogon citrates, Desmodium, Dianthus caryophyllus, Dictamnus albus, Dioscorea bulbifera, Dodonaea viscose, Elytrigia repens, Eryngium foetidum, Glechoma hederacea, Guaiacum sp., Gypsophila acantholimoides, Gypsophila Achaia, Gypsophila acutifolia, Gypsophila adenophora, Gypsophila adenophylla, Gypsophila albida, Gypsophila altissima, Gypsophila antari, Gypsophila antoninae, Gypsophila arabica, Gypsophila aretioides, Gypsophila arrostii, Gypsophila aucheri, Gypsophila aulieatensis, Gypsophila australis, Gypsophila bicolor, Gypsophila capituliflora, Gypsophila cephalotes, Gypsophila cerastioides, Gypsophila davurica, Gypsophila desertorum, Gypsophila elegans, Gypsophila fastigiata, Gypsophila glandulosa, Gypsophila glomerata, Gypsophila huashanensis, Gypsophila imbricate, Gypsophila intricate, Gypsophila iranica, Gypsophila krascheninnikovii, Gypsophila libanotica, Gypsophila licentiana, Gypsophila muralis, Gypsophila nana, Gypsophila oldhamiana, Gypsophila pacifica, Gypsophila paniculata, Gypsophila patrinii, Gypsophila perfoliata, Gypsophila petraea, Gypsophila pilosa, Gypsophila repens, Gypsophila rokejeka, Gypsophila ruscifolia, Gypsophila scorzonerifolia, Gypsophila sericea, Gypsophila silenoides, Gypsophila sp., Gypsophila spinosa, Gypsophila stevenii, Gypsophila struthium, Gypsophila tenuifolia, Gypsophila tschiliensis, Gypsophila uralensis, Gypsophila venusta, Gypsophila viscose, Gypsophila wendelboi, Gypsophila wilhelminae, Gypsophila xanthochlora, Hibiscus sabdariffa, Hyacinthus orientalis, Hyacinthus orientalis, Hydrangea arborescens, Hypericum perforatum, Lagenaria siceraria, Leonurus cardiac, Lonicera japonica, Manilkara zapota, Medicago sativa, Melia azedarach, Menyanthes trifoliate, Ocimum basilicum, Origanum majorana, Panax japonicas, Panax quinquefolius, Panax quinqufolius, Paullinia cupana, Phragmites australis, Polygala tenuifolia, Portulaca oleracea, Quillaja saponaria, Rosa centifolia, Rosa gallica, Rosa laevigata, Ruscus aculeatus, Salvia officinalis, Sapindus delavayi, Sapindus marginatus, Sapindus mukorossi, Sapindus saponaria, Sapindus trifoliatus, Saponaria officinalis, Sida rhombifolia, Smilax spp, Solanum lycopersicum, Solanum melongena, Solanum nigrum, Taraxacum officinale, Terminalia bellirica, Teucrium chamaedrys, Teucrium marum, Teucrium montanum, Teucrium scordium, Tilia sp., Trigonella foenum-graecum, Vinca minor, Viola tricolor, Yucca schidigera, Zea mays, Zizyphus jujube, another plant including saponin, and a combination thereof. This group may be referred to as a saponin plant list.

The saponin plant is a natural plant which includes saponin that can be extracted into water, at least in part. Accordingly, in some embodiments of the invention, the saponin material is obtained by extraction from a plant source by employing water, and in some embodiments, alcohol, glycerin, a water/alcohol solution, or a water/glycerin solution may be used for extraction. The extraction time may vary without limitation from 1 to 8 hours, at or above room temperature (20° C.-30° C.), e.g., above 30° C., 40° C., 50° C., 60° C., or 99° C. In some embodiments, the extraction is carried out at a temperature between 70° C. and 100° C.

In some embodiments, the saponin material is obtained from a plant source. The plant source may be selected from the saponin plant list as described above or any mixture thereof. Any part of the plant may be used for extracting the saponin material, including leaves, stems, roots, bulbs, blossom and fruit (including the skin, flesh and seed of the fruit).

In other embodiments, the saponin-containing extract may be obtained from any natural source known to comprise saponins. Such a natural source may be a plant source, some of which are detailed infra, and also from non-plant sources such as marine organisms (e.g., starfish and sea cucumbers). In some embodiments of the invention, the saponins are extracted from a plant source, naturally grown or genetically modified to have high saponin content.

The extraction medium may be one of an infusion, a decoction, a maceration, or a product from another extraction technique performed on the saponin plant by man. The extraction medium may be produced from an extraction technique on the saponin plant using 1 weight unit of the saponin plant to 5-10 weight units of water.

The composition may further comprise an oil including an essential oil and/or a fixed oil to form an emulsion for transdermal delivery. The oil may be a fixed oil and/or an essential oil of a target plant, the oil having a weight percentage between 0.1 wt % and 6 wt % of the composition. The oil may be from a target plant selected from the group consisting of: Pinus mugo, Rosa damascene, Abies alba, Abies siberica ledeb., Acorus calamus L., Agathosma betulina, Allium sativum L., Amyris balsamifera L., Anethum graveolens, Angelica archangelica L., Aniba rosaeodora Ducke, Anthemis nobilis, Apium graveolens, Artemisia dracunculus L, Artemisia herba-alba Asso, Artemisia pallens Wall., Boswellia carterii, Bulnesia sarmientoi, Cananga odorata, Canarium commune, Carum Carvi L., Cedrus atlantica, Cinnamomum Camphora Ness & Eberm, Cinnamomum cassia, Cinnamomum zeylanicum Blume, Cistus ladaniferus L, Citrus aurantifolia Swingle, Citrus Aurantium, Citrus aurantium amara, Citrus aurantium ssp. Bergamia, Citrus bergamia, Citrus dulcis, Citrus limon, L. Burman, Citrus paradise, Citrus reticulata Blanco, Citrus sinensis, Coleus Forskholii, Commiphora Myrrha, Copaifera officinalis, Coriandrum sativum L, Croton eluteria, Cryptocaryo massoy, Cuminum cyminum L, Cupressus funebris, Cupressus sempervirens L., Cymbopogon flexuosus, Cymbopogon winterianus Jowitt, Daucus Carota Sativa, Elettaria cardamomum L. Maton, Eucalyptus citriodora Hooker, Eucalyptus globulus Labille, Eucalytus radiate, Eugenia caryophyllus, Ferula asafoetida L., Ferula gumosa, Ex-ferula galbaniflua boiss. & buhse, Foeniculum vulgare Miller, Gaultheria procumbens, Helichrysum italicum, Hyssopus officinalis, Illicium verum, Juniperus communis, Juniperus oxycedrus spp, Juniperus virginiana/mexicana, Laurus nobilis, Lavandula angustifolia p. Miller, Lavandula hybrid, Levisticum officinale, Litsea cubeba, Litsea cubeba Pers., Matricaria chamomilla, Matricaria recutita, Melaleuca altemifolia, Melaleuca Leucadendron (L.), Melaleuca Viridiflora, Melissa officinalis, Mentha arvensis, Mentha cardiac, Mentha citrate, Mentha piperita, Mentha pulegium, Mentha spicata, Mentha viridis, Michelia alba, Myristica frangrans, Myrocarpus frondosus, Myrtus communis L., Ocimum basilicum L, Origanum majorana, Origanum vulgare, Pelargonium graveolens, Petroselinum sativum, Pimenta dioica, Pimenta racemosa, Pimpinella anisum, Pinus pumilio, Pinus spp., Pinus sylvestris, Piper nigrum, Pogostemon cablin, Ravensara aromatica, Sonnerat, Rosa centifolia, Rosmarinus officinalis, Salvia hispanica, Salvia officinalis, Salvia sclarea, Santalum album, Santalum austrocaledonicum Vl, Sassafras Albidum M., Satureja hortensis, Satureja Montana, Syzygium aromaticum, Tagetes glandullfera Schrank, Tanacetum Vulgare L., Thuya Occidentalis, Thymus mastichina L., Thymus Serpylum L., Thymus vulgaris, Thymus zygis, Traschispermum ammi, Vetyveria zizanioides, Zingiber officinalis, Prunus amygdalus var. dulcis, Persea gratissima, Prunus armeniaca, Borage officinalis, Camellia japonica, Cocos nucifera, Oenothera biennis, Vitis vinifera, Corylus avellana, Simmondsia chinensis, Aleurites moluccana, Macadamia tetraphylla, Oleo europaea, Rosa Rubiginosa, Sesamum indicum, Helianthus annuus, Tiriticum aestivum, and a combination thereof. This group may be referred to as a target plant list.

The composition may further comprise a preservative providing antioxidant and/or antimicrobial properties. The preservative may be between 0.1 wt % and 3 wt % of the composition. The preservative may be selected from the group consisting of: Acacia ssp, Achillea millefolium, Allium cepa, Allium sativum var. sativum, Aloe spp., Aloe vera, Althaea officinalis, Anethum graveolens, Angelica spp, Apium graveolens, Arachis hypogaea, Artemisia spp, Astragalus membranaceus, Avena sativa, Azadirachta indica, Berberis vulgaris, Beta vulgaris, Borago officinalis, Brassica spp, Camellia sinensis, Capparis spinosa, Capsella bursa-pastoris, Capsicum annuum, Capsicum frutescens, Carum carvi, Castanea sativa, Centella asiatica, Ceratonia siliqua, Chrysanthemum spp, Cinchona spp, Cinnamomum ssp, Citrus ssp, Coffea Arabica, Coriandrum sativum, Crataegus spp, Cucurbita spp, Daucus carota, Digitalis purpurea, Echinacea angustifolia, Echinacea pallid, Elaeagnus angustifolia, Ephedra spp, Eruca sativa, Eucalyptus spp, Ferula spp, Fragaria spp, Garcinia spp, Genista spp, Gentiana lutea, Ginkgo biloba, Glycine max, Glycyrrhiza glabra, Gossypium spp, Hedera helix, Helianthus annuus, Hibiscus rosa-sinensis, Hordeum vulgare, Hydrangea spp, Hypericum perforatum, Inula helenium, Jasminum officinale, Juglans spp, Juniperus spp, Lactuca sativa, Laurus nobilis, Lavandula spp, Lippia spp, Liquidambar orientalis, Lupinus spp, Lycopersicon esculentum, Macadamia spp, Macroptylium spp, Magnolia spp, Malva spp, Melaleuca spp, Melissa officinalis, Mentha spp, Micromeria spp, Morus alba, Myrtus communis, Nasturtium officinale, Nerium oleander, Nigella sativa, Ocimum spp, Oenanthe spp, Olea europaea, Ononis spinosa, Opuntia ficus-indica, Origanum spp, Paeonia spp, Panax spp, Panicum maximum, Passiflora spp, Phaseolus spp, Phyllanthus spp, Physalis spp, Pinus spp, Piper spp, Plantago spp, Podophyllum spp, Polygonum spp, Prunus spp, Punica granatum, Pycnanthemum spp, Quercus spp, Rosa spp, Rosmarinus officinalis, Rubus spp, Rumex spp, Ruscus aculeatus, Ruta graveolens, Salix alba, Salvia spp, Santalum album, Santolina chamaecyparissus, Satureja spp, Schinus molle, Scrophularia spp, Scutellaria spp, Senna spp, Serenoa repens, Sesamum indicum, Sideritis spp, Silybum marianum, Simmondsia chinensis, Smilax spp, Solanum spp, Spirulina spp., Stachys officinalis, Stevia rebaudiana, Styrax benzoin, Syzygium spp, Tagetes spp, Taraxacum officinale, Taxus spp, Thymus spp, Tilia spp, Trifolium spp, Trigonella foenum-graecum, Triticum aestivum, Ulex spp, Uncaria spp, Urtica dioica, Vaccinium spp, Valeriana officinalis, Vanilla planifolia, Verbascum Thapsus, Verbena officinalis, Viburnum opulus, Vinca spp, Viscum album, Vitex agnus-castus, Vitis vinifera, Yucca baccata, Zea mays, Zingiber officinale, and a combination thereof. This group may be referred to as a preservative list.

The composition may further comprise a film-forming biopolymer, wherein the biopolymer is between 0.1 wt % and 4 wt % of the composition. In one example, the film-forming biopolymer may be selected from the group consisting of pullulan or other polysaccharides, galactomannans, Turkish tragacanth, locust bean gum or other gums, alginates, casein or caseinates, and the like. This group may be referred to as a biopolymer list. Advantageously, the film-forming biopolymer functions to form the composition as a film on the application surface, such as skin, to provide an increased length of time for the composition to be absorbed by the application surface.

In accordance with another embodiment, a composition for transdermal delivery comprises: an oily phase including an oil between 0.1 wt % and 6 wt % of the composition; an aqueous phase including an herbal extract solution having soluble components of a subject plant solvated in an extraction medium; and a preservative providing antioxidant and/or antimicrobial properties, wherein the preservative is between 0.1 wt % and 3 wt % of the composition. The herbal extract solution is one of an infusion, a decoction, a maceration, or a product from another extraction technique on the subject plant. The herbal extract solution is produced from an extraction technique on the subject plant using 1 weight unit of the subject plant to 6-12 weight units of the extraction medium. The extraction medium includes soluble components of a saponin plant solvated in water. The extraction medium is produced from an extraction technique on the saponin plant using 1 weight unit of the saponin plant to 5-10 weight units of water. The extraction medium is one of an infusion, a decoction, a maceration, or a product from another extraction technique on the saponin plant. The saponin plant and the subject plant are different types of plants.

The above composition may have the following alternative components, which may also be combined in various applicable and functioning combinations within the scope of the present invention: the subject plant is selected from the group consisting of the subject plant list as described above; the target plant is selected from the group consisting of the target plant list as described above; the saponin plant is selected from the group consisting of the saponin plant list as described above; and the preservative is selected from the group consisting of the preservative list as described above. The composition may further comprise a film-forming biopolymer between 0.1 wt % and 4 wt % of the composition. In one example, the film-forming biopolymer may be selected from the group consisting of the biopolymer list as described above.

Any of the above compositions may further include a component of complimentary function that includes complimentary active ingredients for providing a topical product as desired. The component of complimentary function may be selected from the group consisting of film-forming biopolymers, rheology modifiers, anti-pollution ingredients, UV screeners, hyaluronic acid, ceramides, and humectants including vinegar or betaine. The component of complimentary function may be an oil to form an emulsion. The component of complimentary function may be between 0.1 wt % and 3 wt % of the composition. The component of complimentary function may be a preservative selected from the group consisting of the preservative list as described above.

II. Oil-In-Water Compositions

In accordance with one embodiment, a composition for transdermal delivery comprises an oil in water emulsion having an oil of a target plant dispersed in a dispersion medium, wherein the dispersion medium includes soluble components of a saponin plant solvated in water, and wherein the saponin plant and the target plant are different types of plants.

The above composition may have the following alternative components, which may also be combined in various applicable and functioning combinations within the scope of the present invention.

The oil may include a fixed oil and/or an essential oil of the target plant, the oil having a weight percentage between 0.1 wt % and 6 wt % of the composition.

The target plant may be selected from the group consisting of the target plant list as described above, and a combination thereof.

The saponin plant may be selected from the group consisting of the saponin plant list as described above, another plant including saponin, and a combination thereof.

The saponin plant is a natural plant which includes saponin that can be extracted into water at least in part. Accordingly, in some embodiments of the invention, the saponin material is obtained by extraction from a plant source by employing water, and in some embodiments, alcohol, glycerin or a water/alcohol solution or a water/glycerin solution. The extraction time may vary without limitation from 1 to 8 hours, at or above room temperature (20° C.-30° C.), e.g., above 30° C., 40° C., 50° C., 60° C. or 99° C. In some embodiments, the extraction is carried out at a temperature between 70° C. and 100° C.

In some embodiments, the saponin material is obtained from a plant source. The saponin plant source may be selected from the saponin list as described above or any mixture thereof. Any part of the plant may be used for extracting the saponin material, including leaves, stems, roots, bulbs, blossom and fruit (including the skin, flesh and seed of the fruit).

In other embodiments, the saponin-containing extract may be obtained from any natural source known to comprise saponins. Such natural source may be a plant source, some of which are detailed infra, and also from non-plant sources such as marine organisms (e.g., starfish and sea cucumbers). In some embodiments of the invention, the saponins are extracted from a plant source, naturally grown or genetically modified to have high saponin content.

The dispersion medium is one of an infusion, a decoction, a maceration, or a product from another extraction technique performed on the saponin plant by man. The dispersion medium may be produced from an extraction technique on the saponin plant using 1 weight unit of the saponin plant to 5-10 weight units of water.

The composition may further comprise a preservative. The preservative may be between 0.1 wt % and 3 wt % of the composition. The preservative may be selected from the group consisting of the preservative list as described above.

The composition may further comprise a film-forming biopolymer, wherein the biopolymer is between 0.1 wt % and 4 wt % of the composition. In one example, the film-forming biopolymer may be selected from the group consisting of pullulan or other polysaccharides, galactomannans, Turkish tragacanth, locust bean gum or other gums, alginates, casein or caseinates, and the like. This group may be referred to as a biopolymer list as described above.

In accordance with another embodiment, a composition for transdermal delivery comprises: an oil in water emulsion having a fixed oil and/or an essential oil of a target plant dispersed in a dispersion medium; a preservative providing antioxidant and/or antimicrobial properties, wherein the preservative is between 0.1 wt % and 3 wt % of the composition; and a film-forming biopolymer, wherein the biopolymer is between 0.1 wt % and 4 wt % of the composition. The fixed oil and/or the essential oil of the target plant is between 0.1 wt % and 6 wt % of the composition. The dispersion medium includes soluble components of a saponin plant solvated in water, the dispersion medium is produced from an extraction technique on the saponin plant using 1 weight unit of the saponin plant to 5-10 weight units of water, and the dispersion medium is one of an infusion, a decoction, a maceration, or a product from another extraction technique on the saponin plant. The saponin plant and the target plant are different types of plants.

The above composition may have the following alternative components, which may also be combined in various applicable and functioning combinations within the scope of the present invention: the target plant is selected from the group consisting of the target plant list as described above; the saponin plant is selected from the group consisting of the saponin plant list as described above; the preservative is selected from the group consisting of the preservative list as described above; and the film-forming biopolymer is selected from the group consisting of the biopolymer list as described above.

Any of the above compositions may further include a component of complimentary function that includes complimentary active ingredients for providing a topical product as desired. The component of complimentary function may be selected from the group consisting of film-forming biopolymers, rheology modifiers, anti-pollution ingredients, UV screeners, hyaluronic acid, ceramides, and humectants including vinegar or betaine. The component of complimentary function may be between 0.1 wt % and 3 wt % of the composition. The component of complimentary function may be a preservative selected from the group consisting of the preservative list as described above.

A further understanding of embodiments of the present invention can be obtained by reference to certain specific examples for preparation of extract solutions, extract media, oil dispersions, dispersion media, and/or compositions in accordance with the present invention, which are now provided herein for purposes of illustration only and are not intended to be limiting of the present invention to these examples.

### EXAMPLES

### Extraction Examples: Rosemary

A first rosemary extract solution was prepared from ground rosemary leaves and distilled water in a ratio of 1:5 using decoction technique. This first rosemary extract solution was used as a baseline.

A soapwort extract solution was prepared from ground soapwort roots (i.e., a saponin plant) and distilled water in a ratio of 1:7 using decoction technique to provide a first extraction medium with a saponin content. A second rosemary extract solution was prepared from ground rosemary leaves (subject plant) and the soapwort extract solution (that was prepared beforehand in a ratio of 1:7) in a ratio of 1:5 using decoction technique. This second rosemary extract solution was compared to the first rosemary extract solution.

A licorice (Glycyrrhiza glabra) extract solution was prepared from ground licorice roots (i.e., a saponin plant) and distilled water in a ratio of 1:7 using decoction technique to provide a second extraction medium with a saponin content. A third rosemary extract solution was prepared from ground rosemary leaves (subject plant) and the licorice extract solution (that was prepared beforehand in a ratio of 1:7) in a ratio of 1:5 using decoction technique. This third rosemary extract solution was compared to the first rosemary extract solution.

Analysis of the three rosemary extract solutions showed different extraction media or solvents better extract specific kinds of active materials. High performance liquid chromatography (HPLC) analysis using a Shimadzu Prominence HPLC was conducted for the following active materials: rosmarinic acid, carnosic acid, apigenin, kaempherol, and luteolin. In one example, HPLC settings included a 250×4.6 mm, 5 micron, C18 column, at a column temperature of 30 degrees Celsius, an acetonitrile and formic acid in water mobile phase, and injection volume of 20 microliters. Table 1 below lists HPLC analysis results.

TABLE 1

| | HPLC Analysis Results (mg/L) |
|---|---|
| Rosmarinic Acid Extracted in Water | 31.74 ± 0.57 |
| Rosmarinic Acid Extracted in Licorice | 37.85 ± 0.17 |
| % Extraction Enhancement Potential | 19.25% |
| of Licorice | |
| Carnosic Acid Extracted in Water | 0.48 ± 0.02 |
| Carnosic Acid Extracted in Soapwort | 1.08 ± 0.02 |
| % Extraction Enhancement Potential | 125.00% |
| of Soapwort | |
| Apigenin Extracted in Water | 1.17 ± 0.03 |
| Apigenin Extracted in Soapwort | 7.68 ± 0.02 |
| % Extraction Enhancement Potential | 556.41% |
| of Soapwort | |
| Kaempherol Extracted in Water | 0.00 ± 0.00 |
| Kaempherol Extracted in Licorice | 1.39 ± 0.01 |
| Extraction Enhancement Potential | Significantly higher |
| of Licorice | |
| Luteolin Extracted in Water | 0.15 ± 0.00 |
| Luteolin Extracted in Licorice | 0.36 ± 0.00 |
| % Extraction Enhancement Potential | 140.00% |
| of Licorice | |

Rosmarinic acid was best extracted into the licorice extract solution (second extraction medium), and the rosmarinic acid amount in the licorice extract solution was 20% higher than the rosmarinic acid content that was extracted into water alone. Carnosic acid was best extracted into the soapwort extract solution (first extraction medium), and the carnosic acid amount in the soapwort extract solution was 225% higher than the carnosic acid content that was extracted into water alone. Apigenin was best extracted into the soapwort extract solution (first extraction medium) and the apigenin amount in the soapwort extract solution was 650% higher than the apigenin content that was extracted into water alone. Kaempherol was best extracted into the licorice extract solution (second extraction medium) and the kaempherol amount in the licorice extract solution was significantly higher than the kaempherol content that was extracted into water alone. Luteolin was best extracted into the licorice extract solution (second extraction medium) and the luteolin amount in the licorice extract solution was 240% higher than the luteolin content that was extracted into water alone.

### Transdermal Delivery Example 1: Rosemarinic Acid in Rosemary

The diffusion rates of different rosemary extract solutions through diffusion cells that mimic human skin were calculated and analyzed using UV-VIS spectrophotometry to measure rosmarinic acid content. Franz diffusion cells from Permegear, USA, with 47 mm, 200 nm inert polycarbonate membrane filters, were used with 5 ml extract solution samples. The speed of diffusion of different rosemary extracts were calculated at 1, 2, 3, 4, 10, 20, 30, 60, 120, 360 minutes (and up to 1 day of duration) at a rotational speed of 50 rpm to mimic a dermal application. The amount of rosmarinic acid that passed through the filter was determined by absorbance graphics on a UV spectrophotometer (Agilent 8453). A standard calibration curve was determined for rosmarinic acid and the absorbances were measured at 323 nm to determine the % concentrations released.

For a reference or baseline sample, a decoction of rosemary in water alone was prepared at a 1:5 ratio. Observing the absorbances at 323 nm, at different concentrations of the decoction, 1% of the rosmarinic acid content was found to pass through the membrane after 4 minutes. 100% of the rosmarinic acid content was found to pass through the membrane after 150 minutes.

A soapwort decoction (an extraction medium with a saponin content) was prepared in water at a 1:5 ratio. A consequent decoction of rosemary (i.e., a rosemary extract solution) was prepared in the mentioned soapwort decoction at a 1:5 ratio. For the rosemary that was extracted into the previously prepared soapwort decoction (an extraction medium with a saponin content), rosmarinic acid was found to pass or diffuse significantly more rapidly than in the reference sample. 100% of the rosmarinic acid content was found to pass through the membrane at the end of the 4th minute as compared to the 150^{th} minute of the reference sample. Thus, the transdermal delivery of rosmarinic acid using the method of the present invention is much faster than classical water decoctions predominantly used in modern dermal or cosmetic formulations.

### Transdermal Delivery Example 2: Rosemarinic Acid in Rosemary

The diffusion rates of different rosemary extract solutions through diffusion cells that mimic human skin were calculated and analyzed using UV-VIS spectrophotometry to measure rosmarinic acid content. Franz diffusion cells from Permegear, USA, with 47 mm, 200 nm inert polycarbonate membrane filters, were used with 5 ml extract solution samples. The speed of diffusion of different rosemary extracts were calculated at 1, 2, 3, 5, 40 minutes (and up to 1 day of duration) at a rotational speed of 50 rpm to mimic a dermal application. The amount of rosmarinic acid that passed through the filter was determined by absorbance graphics on a UV spectrophotometer (Agilent 8453). A standard calibration curve was determined for rosmarinic acid and the absorbances were measured at 323 nm to determine the % concentrations released.

A soapwort decoction (an extraction medium with a saponin content) was prepared in water at a 1:5 ratio. A decoction of rosemary (i.e., a rosemary extract solution) was prepared in 5×distilled water. The mentioned soapwort decoction and the water decoction of the rosemary leaves were then mixed at a 1:1 ratio. The following diffusion values were measured for this mixture: 14% at minute 1, 49.4% at minute 3, and 100% at minute 5.

100% of the rosmarinic acid content was found to pass through the membrane at the end of the 5th minute as compared to the 150^{th} minute of the reference sample of Transdermal Delivery Example 1 above. Thus, later addition of soapwort extract (the saponin extract decoction was not used as an extraction medium but was added later to the normal water extract of the herb) decreases transdermal delivery time of rosmarinic acid (as compared to the reference sample of a water decoction of rosemary) from 150 minutes to 5 minutes, a 1/30 fraction of the control sample diffusion time. It was surprisingly found that a decoction of the said content extracted directly into a soapwort decoction had a penetration rate that was approximately 30× the rate of the control sample of only water decoction of rosemary.

### Transdermal Delivery Example 3: Rosemarinic Acid in Rosemary Oil

The diffusion rates of rosemarinic acid in rosemary essential oil through diffusion cells that mimic human skin were calculated and analyzed using UV-VIS spectrophotometry to measure rosmarinic acid content. Franz diffusion cells from Permegear, USA, with 47 mm, 200 nm inert polycarbonate membrane filters, were used with 5 ml extract solution samples. The speed of diffusion of different rosemary extracts were calculated at 1, 2, 3, 4, 7, 10, 20, 40 minutes (and up to 1 day of duration) at a rotational speed of 50 rpm to mimic a dermal application. The amount of rosmarinic acid that passed through the filter was determined by absorbance graphics on a UV spectrophotometer (Agilent 8453). A standard calibration curve was determined for rosmarinic acid and the absorbances were measured at 323 nm to determine the % concentrations released.

Based on the absorbance values, 0% of the rosemarinic acid was released at the end of 40 minutes. This indicated that rosemarinic acid alone was not released through the Franz diffusion cells. Absorbances and diffusion rates were measured up to 48 hours and minimal absorbance values were measured at the end of 48 hours. This indicates that the transdermal delivery of rosemarinic acid in rosemary essential oil alone is negligible and so rosemarinic acid in rosemary oil alone would remain on the skin surface with a potential of causing irritation.

### Transdermal Delivery Example 4: Carvacrol in Thyme Oil

The diffusion rates of carvacrol in thyme (Tymus vulgaris) essential oil through diffusion cells that mimic human skin were calculated and analyzed using UV-VIS spectrophotometry to measure carvacrol content. Franz diffusion cells from Permegeaer, USA, with 47 mm, 200 nm inert polycarbonate membrane filters, were used with 5 ml extract solution samples. The speed of diffusion of different thyme extracts were calculated at 1, 2, 3, 4, 7, 10, 20, 40 minutes (and up to 1 day of duration) at a rotational speed of 50 rpm to mimic a dermal application. The amount of carvacrol that passed through the filter was determined by absorbance graphics on a UV spectrophotometer (Agilent 8453). A standard calibration curve was determined for carvacrol and the absorbances were measured at 272 nm to determine the % concentrations released.

Based on the absorbance values, 0% of the carvacrol was released at the end of 40 minutes. This indicated that carvacrol alone was not released through the Franz diffusion cells. Absorbances and diffusion rates were measured up to 48 hours and minimal absorbance values were measured at the end of 48 hours. This indicates that the transdermal delivery of carvacrol in thyme essential oil alone is negligible and so carvacrol in thyme essential oil alone would remain on the skin surface with a potential of causing irritation.

### Transdermal Delivery Example 5: Carvacrol in Thyme Oil

The diffusion rates of carvacrol in thyme (Tymus vulgaris) essential oil (0.05 ml of thyme essential oil) dispersed in 1 ml of soapwort extract solution through diffusion cells that mimic human skin were calculated and analyzed using UV-VIS spectrophotometry to measure carvacrol content. Franz diffusion cells from Permegear, USA, with 47 mm, 200 nm inert polycarbonate membrane filters, were used with 5 ml extract solution samples. The speed of diffusion of different thyme extracts were calculated at 1, 3, 5, 7, 10, 20, 40 minutes (and up to 1 day of duration) at a rotational speed of 50 rpm to mimic a dermal application. The amount of carvacrol that passed through the filter was determined by absorbance graphics on a UV spectrophotometer (Agilent 8453). A standard calibration curve was determined for carvacrol and the absorbances were measured at 272 nm to determine the % concentrations released.

Based on the absorbances measured, the following diffusion values were determined: 8.20% at minute 1, 26.43% at minute 3, 44.75% at minute 5, 66.29% at minute 7, 77.15% at minute 10, 91.39% at minute 20, and 100% at minute 40.

Thus, 100% of the carvacrol in thyme essential oil in soapwort extract solution was released at the end of 40 minutes as compared to the negligible transdermal delivery of carvacrol in thyme essential oil alone. In other words, when thyme essential oil was not dispersed in water or in another carrier fluid (pure thyme essential oil), the active ingredient carvacrol had negligible penetration, whereas thyme essential oil dispersed in the extraction medium had 100% penetration in 40 minutes.

### Transdermal Delivery Example 6: Carvacrol in Thyme Oil

The diffusion rates of carvacrol in thyme (Tymus vulgaris) essential oil (0.05 ml of thyme essential oil dispersed in 1 ml of licorice extract solution) through diffusion cells that mimic human skin were calculated and analyzed using UV-VIS spectrophotometry to measure carvacrol content. Franz diffusion cells from Permegeaer, USA, with 47 mm, 200 nm inert polycarbonate membrane filters, were used with 5 ml extract solution samples. The speed of diffusion of different thyme extracts were calculated at 1, 2, 3, 4, 7, 10, 20, 30, 40 minutes (and up to 1 day of duration) at a rotational speed of 50 rpm to mimic a dermal application. The amount of carvacrol that passed through the filter was determined by absorbance graphics on a UV spectrophotometer (Agilent 8453). A standard calibration curve was determined for carvacrol and the absorbances were measured at 272 nm to determine the % concentrations released.

Based on the absorbances measured, the following diffusion values were determined:

0% at minute 1, 3.98% at minute 2, 14.41% at minute 3, 32.98% at minute 4, 42.59% at minute 7, 56.35% at minute 10, 67.61% at minute 20, 77.73% at minute 30, and 90.98% at minute 40.

Thus, 91% of the carvacrol in thyme essential oil in licorice extract solution was released at the end of 40 minutes as compared to the negligible transdermal delivery of carvacrol in thyme essential oil alone. The diffusion rates were studied for 24 hours and 100% diffusion was reached after 24 hours.

100% of the carvacrol present in soapwort extract solution was released at the end of 40 minutes whereas about 90% of carvacrol present in licorice extract solution at the end of 40 minutes. When the solubilities were compared, it was observed that 0.5% of thyme oil was clearly dissolved in soapwort water extract and maximum 2% of thyme oil could be homogenously dispersed in soapwort. The maximum amount of thyme oil dissolved in licorice was 0.5%. Absorbance results clearly indicate that soapwort water extract has higher potential to dissolve and diffuse carvacrol of Thyme essential oil than does licorice.

### Example Conclusions

Overall, it has been shown that a decoction of licorice (licorice extract solution) or soapwort (soapwort extract solution) has better extracted rosmarinic acid from rosemary in comparison to plain DIW. Likewise, transdermal delivery of rosmarinic acid from rosemary is modeled to have much improved diffusion with a soapwort extract solution or licorice extract solution compared to that of rosemary (and rosmarinic acid) in water alone without the soapwort or licorice extract solutions.

Combined extraction and transdermal delivery capacity significantly increases when some plant ingredients are extracted and transdermally delivered with the use of a saponin plant, such as soapwort and/or licorice. Similar extraction and transdermal delivery results can be achieved by using other saponin containing plants such as those listed above.

Products of Composition

In accordance with one embodiment, a product is provided comprising any of a composition as described in the various embodiments above. The product may be selected from the group consisting of a cosmetic, a preservative formulation, an antimicrobial formulation, a pharmaceutical composition, a medical device, and a disinfectant.

For example, the present invention provides a cosmetic or cleansing formulation comprising a composition as described in the various embodiments above. The cosmetic or cleansing formulations according to the invention are typically formulated in a form adapted for topical application comprising a cosmetically or dermatologically acceptable medium, namely a medium which is suitable for application onto the skin of a subject (human or non-human). The medium may be in the form of aqueous or hydroalcoholic solution, an oil-in-water or water-in-oil emulsion, a microemulsion, aqueous or anhydrous gels, serum, or else a dispersion of vesicles, a patch, cream, spray, salve, ointment, lotion, gel, solution, suspension, or any other known cosmetically acceptable form. The formulation may alternatively be formulated for application to the human skin (including mucosal regions via a mucous membrane or mucosa), hair, eyelashes, eyebrows, or nails.

In addition, the formulation may contain other standard additives such as an emollient, moisturizer, thickener, emulsifier, neutralizer, coloring agent, a fragrance, absorber or filter, preservative and/or gelling agent, a sun screen agent (e.g., UV absorbing agents), electrolytes, proteins, antioxidants, anti-pollution additives and chelating agents.

The formulation may also further comprise at least one active ingredient such as peptide active ingredients, vegetable extracts, anti-age agents, anti-wrinkle agents, soothing agents, radical scavengers, UV absorbing agents, agents stimulating the synthesis of dermal macromolecules or the energy metabolism, hydrating agents, anti-bacterial agents, anti-fungal agents, anti-inflammatory agents, anesthetic agents, agents modulating cutaneous differentiation, pigmentation or de-pigmentation, agents stimulating nail or hair growth.

In a further aspect, the invention provides an antimicrobial formulation comprising a composition as described in the various embodiments above. The antimicrobial formulation of the invention may be effective in reducing or eliminating a microorganism population or a biofilm of such microorganisms. According to some embodiments, the antimicrobial formulations of the invention are effective in reducing, inhibiting, eliminating, and/or preventing the growth of bacteria, fungi, yeast, viruses and/or other microbes. Such formulations may also contain cell wall permeabilizers for the targeted microbes selected from EDTA, Chitosan or other cationic surfactants.

In a further aspect, the invention provides a therapeutic formulation (pharmaceutical composition) comprising a composition as described in the various embodiments above or a mixture of extracts thereof, as defined herein.

The pharmaceutical formulation of the invention may be effective in the treatment and/or prevention of a variety of diseases and disorders. The formulations of the invention may provide instant and persistent antimicrobial activity against a wide spectrum of microorganisms, as defined herein. In some embodiments, the disease or disorder to be treated is associated with bacterial infection, fungal infection, or viral infection.

In another aspect of the invention, there is provided the use of a formulation of the invention as herein defined, for the preparation of a pharmaceutical composition for treating or preventing a disease or disorder in a mammal (human or non-human). In some embodiments, the disease or disorder is associated with a bacteria, virus, fungus, yeast, or mold.

As used herein, the term “treatment” refers to the administering of a therapeutic amount of the composition of the present invention which is effective to ameliorate undesired symptoms associated with a disease, to prevent the manifestation of such symptoms before they occur, to slow down the progression of the disease, slow down the deterioration of symptoms, to enhance the onset of remission period, slow down the irreversible damage caused in the progressive chronic stage of the disease, to delay the onset of said progressive stage, to lessen the severity or cure the disease, to improve survival rate or more rapid recovery, or to prevent the disease from occurring, or a combination of two or more of the above. The “effective amount” for purposes disclosed herein is determined by such considerations as may be known in the art. The amount must be effective to achieve the desired therapeutic effect as described above, depending, inter alia, on the type and severity of the disease to be treated and the treatment regime. The effective amount is typically determined in appropriately designed clinical trials (dose range studies) and the person versed in the art will know how to properly conduct such trials in order to determine the effective amount. As generally known, an effective amount depends on a variety of factors including the affinity of the ligand to the receptor, its distribution profile within the body, a variety of pharmacological parameters such as half life in the body, on undesired side effects, if any, on factors such as age, gender, etc.

In yet another aspect, the invention provides a preservative formulation comprising a composition as described in the various embodiments above or a mixture of extracts thereof, as defined herein. The preservative formulation of the invention may be used to reduce, inhibit or completely eliminate pathogen population in a variety of consumer products, such as personal care products, industrial products, food products, therapeutics, and others. The formulation of the invention may be used to replace currently available chemicals which are used as preservatives, some of which known as toxic to humans and animals, or at reduce their concentration in such products for human or animal use. The preservative formulation may be added to any such product, such as cosmetics and toiletries in aqueous or hydroalcoholic solution, oil-in-water or water-in-oil emulsion, aqueous or anhydrous gels, cream, ointment, lotion, gel, solution and suspension; therapeutics and over-the-counter pharmaceutical products.

Methods of Enhancing Transdermal Delivery

In accordance with one embodiment, a method for treating human skin is provided, the method comprising applying, to a skin surface, any of a composition as described in various embodiments above.

I. Methods of Enhancing Herbal Extraction and Transdermal Delivery

In accordance with yet another embodiment, a method for enhanced herbal extraction and transdermal delivery of an herbal extract solution comprises: extracting soluble components of a saponin plant with water to provide an extraction medium; extracting soluble components of a subject plant with the extraction medium to provide an herbal extract solution; and mixing the herbal extract solution with a component of complimentary function to produce a product for transdermal delivery.

The above method may have the following alternative components or steps, which may also be combined in various applicable and functioning combinations within the scope of the present invention:

The subject plant may be selected from the group consisting of the subject plant list as described above. The saponin plant may be selected from the group consisting of the saponin plant list as described above. The oil may be a fixed oil or an essential oil of a target plant, and the target plant may be selected from the group consisting of the target plant list as described above.

The extracting of soluble components of the subject plant with the extraction medium may be accomplished by one of an infusion, a decoction, a maceration, or another extraction technique performed on the subject plant by man.

The extracting of soluble components of a saponin plant with water may be accomplished by one of an infusion, a decoction, a maceration, or another extraction technique performed on the saponin plant by man. The extracting of soluble components of a saponin plant with water may include using 1 weight unit of the saponin plant to 5-10 weight units of water.

The component of complimentary function includes complimentary active ingredients for providing a topical product as desired. The component of complimentary function may be selected from the group consisting of film-forming biopolymers, rheology modifiers, anti-pollution ingredients, UV screeners, hyaluronic acid, ceramides, and humectants including vinegar or betaine. The component of complimentary function may be an oil to form an emulsion. The component of complimentary function may be between 0.1 wt % and 3 wt % of the composition. The component of complimentary function may be a preservative selected from the group consisting of the preservative list as described above.

The method may further comprise processing a mixture of the subject plant and the extraction medium prior to the step of extracting soluble components of the subject plant, wherein processing the mixture of the subject plant and the extraction medium includes one of mixing, heating, cavitation, application of direct electric current, ultrasound mixing, and a combination thereof.

The method may further comprise processing a mixture of the saponin plant and water prior to the step of extracting soluble components of the saponin plant, wherein processing the mixture of the saponin plant and water includes one of mixing, heating, cavitation, application of direct electric current, ultrasound mixing, and a combination thereof.

According to alternative embodiments, extraction enhancement may be accomplished by the following methods among others. In accordance with one embodiment, soluble components of a subject plant are extracted into water by decoction, infusion, maceration, or another extraction technique. Then, an extraction medium including saponin (e.g., an aqueous solution including extract of a saponin-containing plant) is added to the extract solution of the subject plant to enhance extraction of soluble components from the subject plant. In accordance with another embodiment, soluble components of a subject plant are extracted directly into an extraction medium including saponin by decoction, infusion, maceration, or another extraction technique. In accordance with yet another embodiment, oil soluble ingredients of a subject plant that exist as an essential or fixed oil, are dispersed in a dispersion medium including saponin, to thereby enhance emulsion of the oil soluble ingredients and to protect the oil from microbial and/or oxidative deterioration.

Although the present invention and disclosure are not limited to this theory, it is believed the extraction of natural ingredients is enhanced significantly by the permeabilization or rupture of cell walls and cell membranes by natural extraction enhancers of the present disclosure (extraction media including saponin). Cell rupture enhances extraction, and/or can enhance antibacterial, antiviral, and/or antifungal effects of a co-existing preservative by membrane rupture of pathogenic organisms. Advantageously, these natural extraction enhancers cause no or negligible negative effects in the targeted areas of the body. Extracted natural ingredients can be delivered into relevant levels of skin or other parts of the body by the employment of transdermals or cell membrane permeabilization agents that are called delivery agents. In the integrated method of the present disclosure, products that have both properties, as an extraction enhancer as well as a delivery agent, are employed.

It is noted that the herbal extract infusion may be used without separation from herbal residue, or the herbal residue after physical filtering may be used without separation from the extraction medium. Furthermore, an herbal extract solution enables the incorporation of desired ingredients of plants into the product without the need for an external solvent and subsequent solvent removal.

The present invention eliminates the extra step needed to separate an extract from a solvent used in the extraction process, as the solvent itself is a raw material of the end product that is produced. This also eliminates the risk of degradation of the active ingredients in the extracts since high temperatures for solvent separation are avoided. Extracts that are produced in the production area in necessary amounts when needed are more fresh and potent than those produced by other methods or requiring lengthy storage times.

Thus, the present invention provides for production in simpler extraction facilities with lower costs of investment, stock and operations when utilizing herbal extracts in the production of soap, household, cosmetics, and like products. For example, herbal extraction as described above may take place in the production area.

II. Methods of Enhancing Oil Dispersion and Transdermal Delivery

In accordance with yet another embodiment, a method for enhanced oil dispersion and transdermal delivery of an oil comprises: extracting soluble components of a saponin plant with water to provide a dispersion medium; dispersing an oil in the dispersion medium to provide an emulsion; and mixing the emulsion with a component of complimentary function to produce a product for transdermal delivery.

The above method may have the following alternative components or steps, which may also be combined in various applicable and functioning combinations within the scope of the present invention.

The saponin plant may be selected from the group consisting of the saponin plant list as described above. The oil may be a fixed oil and/or an essential oil of a target plant, and the target plant may be selected from the group consisting of the target plant list as described above. The component of complimentary function may be a preservative selected from the group consisting of the preservative list as described above or a film forming biopolymer selected from the group consisting of the biopolymer list as described above.

The extracting of soluble components of a saponin plant with water may include using 1 weight unit of the saponin plant to 5-10 weight units of water.

The extracting of soluble components of the saponin plant with water may be accomplished by one of an infusion, a decoction, a maceration, or another extraction technique performed on the saponin plant by man.

The oil may be between 0.1 wt % and 6 wt % of the composition.

The component of complimentary function may be selected from the group consisting of film-forming biopolymers, rheology modifiers, anti-pollution ingredients, UV screeners, hyaluronic acids, ceramides, and humectants including vinegar or betaine. The component of complimentary function may be between 0.1 wt % and 4 wt % of the composition.

The method may further comprise processing a mixture of the saponin plant and water prior to the step of dispersing an oil in the dispersion medium, wherein processing the mixture of the saponin plant and water includes one of mixing, heating, cavitation, application of direct electric current, ultrasound mixing, and a combination thereof.

The foregoing examples have been provided merely for the purpose of explanation and are in no way to be construed as limiting of the present invention. While the present invention has been disclosed with reference to embodiments, the words used herein are intended to be words of description and illustration, rather than words of limitation. While the present invention has been described with reference to particular materials and embodiments, the present invention is not intended to be limited to the particulars disclosed herein. Rather, the present invention extends to all functionally equivalent structures, materials, and uses, such as are within the scope of the appended claims. Changes may be made, within the purview of the appended claims, as presently stated and as may be amended, without departing from the scope and spirit of the present invention. All terms used in this disclosure should be interpreted in the broadest possible manner consistent with the context.

1-35. (canceled)

36. A composition for transdermal delivery, comprising:
an herbal extract solution having soluble components of a subject plant solvated in an extraction medium;
wherein the extraction medium includes soluble components of a saponin plant solvated in water, and
wherein the saponin plant and the subject plant are different types of plants.

37. The composition according to claim 36, wherein the subject plant is selected from the group consisting of Achillea Millefolium, Aesculus Hippocastanum, Agrimonia Aupatoria, Akhemilla Vulgaris, Allium Sativum, Althaea Officinalis, Angelica Archangelica, Angelica sinensis syn. A. polymorpha, Anthemis Nobilis, Apium Graveolens, Arctostaphylos Uva Ursi, Arnica Montana, Artemisia Vulgaris, Articum Lappa, Aspalathus Linearis, Astragalus membranaceus, Berberis Vulgaris, Betula Alba, Borago Officinalis, Calendula Officincalis, Calluna Vulgaris, Camellia Sinensis, Capsella Bursa Pastoris, Capsicum Annuum, Cardus Benedictus, Cardus Marianus, Carum Carvi, Cassia Angustifolia, Centella asiatica syn. Hydrocotyle, Ceranus Vulgaris, Ceratonia siliqua, Chamaecrista mimosoides (L.), Cichorium Intybus, Cimicifuga racemosa, Cinnamomum Zeylanicum, Citrus paradisi Macfadyen, Citrus Aurantium, Citrus Limonum, Citrus Sinensis, Cnidium monnieri, Coffea Cruda, Cordyceps sinensis (Berk.) Sacc., Cortex Magnoliae Officinalis, Corylus Avellana, Crataegus monogyna, Crataegus cuneata Sieb. et Zucc., Crataegus Oxyacantha, Cucurbita Pepo, Cupressus Sempervirens, Curcuma Longa, Cymbopogon Flexuosus, Cyminum Cuminum, Cynara Scolymus, Daucus Carota, Dioscoreavillosa, Echinacea Angustifolia, Echinacea Purpurea, Elettaria Cardamomum, Eleutherococcus Senticosus, Eleutherococcus senticosus, Acanthopanax senticosus, Epimedium brevicornum, Equisetum Arvense, Erigeron Canadensis, Eschscholtzia Californica, Eucalyptus Globulus, Ferula elaeochytris, Foeniculum Vulgare, Fragaria Vesca, Fraxinus Exelsior, Fructus Schisandrae, Fumaria Officinalis, Galla Chinensis, Gentiana Lutea, Ginkgo Biloba, Glechoma Hederaceum, Glycine max, Glycyrrhiza Glabra, Hamamelis Virginiana, Harpagophytum, Hibiscus Sabdariffa, Hieracium Pilosella, Humulus Lupulus, Hypericum Perforatum, Hysopus Officinalis, Juglans Regia, Juniperus Communis, Lavandula Angustifolia, Levisticum Officinalis, Ligustrum lucidum, Aitor Eriobotrya japonica (Thunb) Lindl. L., Lippia Citriodora, Lotus Corniculatus, Lycium Barbarum, Lycopersicum esculentum, Malva Sylvestris, Matricaria Chamomilla, Medicago Sativa, Melilotus Officinalis, Melissa Officinalis, Mentha Piperita, Mentha Viridis, Momordica Charantia, Ocimum Basilicium, Olea Europea, Ononis Spinosa, Origanum Majorana, Origanum Vulgaris, Paeonia Albiflora Root, Paeonia suffruticosa, Passiflora Incarnata, Passiflora quadrangularis, Paullinia Cupana, Perilla frutescens (L.) Britton., Phaseolus Vulgaris, Pimpinella Anisum, Pinus Pinaster, Pinus strobes, Pinus Sylvestris, Plantago Lanceolata, Plantago Major, Pneumus Boldus, Polygonum cuspidatum sieb. et zucc, Primula Officinalis, Pueraria lobata (Willd.) Ohwi, Pulmonaria Officinalis, Punica granatum L., Punica Granatum, Pyrus malus, Quercus Robur, Quercus spp., Raphanus Sativus, Rhamnus Frangula, Rheum Officinalis, Rhodiola rosea L., Ribers Nigrum, Rosa Canina, Rosmarinus Officinalis, Rubus fructicosus, Rubus Ideaus, Ruscus Aculeatus, Sabal Serrulata, Salix Alba, Salvia miltiorrhiza, Salvia Officinalis, Salvia Sclarea, Sambucus Nigra, Satureja Montana, Serenoa repens, Silybum marianum (L.) Gaertner, Soja Hispida, Solidago Virga Aurea, Spinacia Oleracea, Spirea Ulmaria, Symphytum officinalis, Taraxacum officinale, Taraxacum Officinalis, Thymus sp., Thymus vulgaris, Tilia Officinalis, Tribulus terrestris, Trigonella, Uncaria Tomentosa, Urtica Dioica, Valeriana Officinalis, Verbascum Thapsus, Verbena Officinalis, Viburnum Lanta, Viola Tricolor, Viscum Album, Vitex Agnus Castus, Vitis Vinifera, Zea Mais, Zingiber Officinalis, and a combination thereof.

38. The composition according to claim 36, wherein the saponin plant is selected from the group consisting of Camellia sinensis, Styrax japonica, Acacia concinna, Acacia nilotica, Acorus calamus, Aesculus hippocastanum, Agave Americana, Ailanthus altissima, Akebia quinata, Albizia julibrissin, Aletris farinose, Aleurites fordii, Allium cepa, Allium drummondii, Allium fistulosum, Allium neapolitanum, Allium oleraceum, Allium ramosum, Allium sativum var. sativum, Allium schoenoprasum, Allium sphaerocephalon, Allium tricoccum, Allium tuberosum, Allium ursinum, Allium vineale, Anemone pulsatilla, Arisaema consanguineum, Asparagus lucidus, Balanites aegyptiacus, Banisteriopsis caapi, Bupleurum falcatum, Calendula officinalis, Camellia oleifera, Capparis spinosa, Capsicum annuum, Castanospermum austral, Caulophyllum thalictroides, Centella asiatica, Ceratonia siliqua, Chrysophyllum cainito, Cissampelos pareira, Coix lacryma-jobi, Colocasia esculenta, Colubrina arborescens, Corchorus olitorius, Cymbopogon citrates, Desmodium, Dianthus caryophyllus, Dictamnus albus, Dioscorea bulbifera, Dodonaea viscose, Elytrigia repens, Eryngium foetidum, Glechoma hederacea, Guaiacum sp., Gypsophila acantholimoides, Gypsophila Achaia, Gypsophila acutifolia, Gypsophila adenophora, Gypsophila adenophylla, Gypsophila albida, Gypsophila altissima, Gypsophila antari, Gypsophila antoninae, Gypsophila arabica, Gypsophila aretioides, Gypsophila arrostii, Gypsophila aucheri, Gypsophila aulieatensis, Gypsophila australis, Gypsophila bicolor, Gypsophila capituliflora, Gypsophila cephalotes, Gypsophila cerastioides, Gypsophila davurica, Gypsophila desertorum, Gypsophila elegans, Gypsophila fastigiata, Gypsophila glandulosa, Gypsophila glomerata, Gypsophila huashanensis, Gypsophila imbricate, Gypsophila intricate, Gypsophila iranica, Gypsophila krascheninnikovii, Gypsophila libanotica, Gypsophila licentiana, Gypsophila muralis, Gypsophila nana, Gypsophila oldhamiana, Gypsophila pacifica, Gypsophila paniculata, Gypsophila patrinii, Gypsophila perfoliata, Gypsophila petraea, Gypsophila pilosa, Gypsophila repens, Gypsophila rokejeka, Gypsophila ruscifolia, Gypsophila scorzonerifolia, Gypsophila sericea, Gypsophila silenoides, Gypsophila sp., Gypsophila spinosa, Gypsophila stevenii, Gypsophila struthium, Gypsophila tenuifolia, Gypsophila tschiliensis, Gypsophila uralensis, Gypsophila venusta, Gypsophila viscose, Gypsophila wendelboi, Gypsophila wilhelminae, Gypsophila xanthochlora, Hibiscus sabdariffa, Hyacinthus orientalis, Hyacinthus orientalis, Hydrangea arborescens, Hypericum perforatum, Lagenaria siceraria, Leonurus cardiac, Lonicera japonica, Manilkara zapota, Medicago sativa, Melia azedarach, Menyanthes trifoliate, Ocimum basilicum, Origanum majorana, Panax japonicas, Panax quinquefolius, Panax quinqufolius, Paullinia cupana, Phragmites australis, Polygala tenuifolia, Portulaca oleracea, Quillaja saponaria, Rosa centifolia, Rosa gallica, Rosa laevigata, Ruscus aculeatus, Salvia officinalis, Sapindus delavayi, Sapindus marginatus, Sapindus mukorossi, Sapindus saponaria, Sapindus trifoliatus, Saponaria officinalis, Sida rhombifolia, Smilax spp, Solanum lycopersicum, Solanum melongena, Solanum nigrum, Taraxacum officinale, Terminalia bellirica, Teucrium chamaedrys, Teucrium maxum, Teucrium montanum, Teucrium scordium, Tilia sp., Trigonella foenum-graecum, Vinca minor, Viola tricolor, Yucca schidigera, Zea mays, Zizyphus jujube, another plant including saponin, and a combination thereof.

39. The composition according to claim 36, wherein the extraction medium is produced from an extraction technique on the saponin plant using 1 weight unit of the saponin plant to 5-10 weight units of water.

40. The composition according to claim 36, further comprising an oil including an essential oil and/or a fixed oil to form an emulsion for transdermal delivery.

41. The composition according to claim 40, wherein the oil is from a target plant selected from the group consisting of Pinus mugo, Rosa damascene, Abies alba, Abies siberica ledeb., Acorus calamus L., Agathosma betulina, Allium sativum L., Amyris balsamifera L., Anethum graveolens, Angelica archangelica L., Aniba rosaeodora Ducke, Anthemis nobilis, Apium graveolens, Artemisia dracunculus L, Artemisia herba-alba Asso, Artemisia pallens Wall., Boswellia carterii, Bulnesia sarmientoi, Cananga odorata, Canarium commune, Carum Carvi L., Cedrus atlantica, Cinnamomum Camphora Ness & Eberm, Cinnamomum cassia, Cinnamomum zeylanicum Blume, Cistus ladaniferus L, Citrus aurantifolia Swingle, Citrus Aurantium, Citrus aurantium amara, Citrus aurantium ssp. Bergamia, Citrus bergamia, Citrus dulcis, Citrus limon, L. Burman, Citrus paradise, Citrus reticulata Blanco, Citrus sinensis, Coleus Forskholii, Commiphora Myrrha, Copaifera officinalis, Coriandrum sativum L, Croton eluteria, Cryptocaryo massoy, Cuminum cyminum L, Cupressus funebris, Cupressus sempervirens L., Cymbopogon flexuosus, Cymbopogon winterianus Jowitt, Daucus Carota Sativa, Elettaria cardamomum L. Maton, Eucalyptus citriodora Hooker, Eucalyptus globulus Labille, Eucalytus radiate, Eugenia caryophyllus, Ferula asafoetida L., Ferula gumosa, Ex-ferula galbaniflua boiss. & buhse, Foeniculum vulgare Miller, Gaultheria procumbens, Helichrysum italicum, Hyssopus officinalis, Illicium verum, Juniperus communis, Juniperus oxycedrus spp, Juniperus virginiana/mexicana, Laurus nobilis, Lavandula angustifolia p. Miller, Lavandula hybrid, Levisticum officinale, Litsea cubeba, Litsea cubeba Pers., Matricaria chamomilla, Matricaria recutita, Melaleuca alternifolia, Melaleuca Leucadendron (L.), Melaleuca Viridiflora, Melissa officinalis, Mentha arvensis, Mentha cardiac, Mentha citrate, Mentha piperita, Mentha pulegium, Mentha spicata, Mentha viridis, Michelia alba, Myristica frangrans, Myrocarpus frondosus, Myrtus communis L., Ocimum basilicum L, Origanum majorana, Origanum vulgare, Pelargonium graveolens, Petroselinum sativum, Pimenta dioica, Pimenta racemosa, Pimpinella anisum, Pinus pumilio, Pinus spp., Pinus sylvestris, Piper nigrum, Pogostemon cablin, Ravensara aromatica, Sonnerat, Rosa centifolia, Rosmarinus officinalis, Salvia hispanica, Salvia officinalis, Salvia sclarea, Santalum album, Santalum austrocaledonicum Vl, Sassafras Albidum M, Satureja hortensis, Satureja Montana, Syzygium aromaticum, Tagetes glandullfera Schrank, Tanacetum Vulgare L., Thuya Occidentalis, Thymus mastichina L., Thymus Serpylum L., Thymus vulgaris, Thymus zygis, Traschispermum ammi, Vetyveria zizanioides, Zingiber officinalis, Prunus amygdalus var. dulcis, Persea gratissima, Prunus armeniaca, Borage officinalis, Camellia japonica, Cocos nucifera, Oenothera biennis, Vitis vinifera, Corylus avellana, Simmondsia chinensis, Aleurites moluccana, Macadamia tetraphylla, Oleo europaea, Rosa Rubiginosa, Sesamum indicum, Helianthus annuus, Tiriticum aestivum, and a combination thereof.

42. The composition according to claim 41, wherein the oil is a fixed oil and/or an essential oil of a target plant, the oil having a weight percentage between 0.1 wt % and 6 wt % of the composition.

43. The composition according to claim 36, further comprising a preservative providing antioxidant and/or antimicrobial properties.

44. The composition according to claim 43, wherein the preservative is selected from the group consisting of Acacia ssp, Achillea millefolium, Allium cepa, Allium sativum var. sativum, Aloe spp., Aloe vera, Althaea officinalis, Anethum graveolens, Angelica spp, Apium graveolens, Arachis hypogaea, Artemisia spp, Astragalus membranaceus, Avena sativa, Azadirachta indica, Berberis vulgaris, Beta vulgaris, Borago officinalis, Brassica spp, Camellia sinensis, Capparis spinosa, Capsella bursa-pastoris, Capsicum annuum, Capsicum frutescens, Carum carvi, Castanea sativa, Centella asiatica, Ceratonia siliqua, Chrysanthemum spp, Cinchona spp, Cinnamomum ssp, Citrus ssp, Coffea Arabica, Coriandrum sativum, Crataegus spp, Cucurbita spp, Daucus carota, Digitalis purpurea, Echinacea angustifolia, Echinacea pallid, Elaeagnus angustifolia, Ephedra spp, Eruca sativa, Eucalyptus spp, Ferula spp, Fragaria spp, Garcinia spp, Genista spp, Gentiana lutea, Ginkgo biloba, Glycine max, Glycyrrhiza glabra, Gossypium spp, Hedera helix, Helianthus annuus, Hibiscus rosa-sinensis, Hordeum vulgare, Hydrangea spp, Hypericum perforatum, Inula helenium, Jasminum officinale, Juglans spp, Juniperus spp, Lactuca sativa, Laurus nobilis, Lavandula spp, Lippia spp, Liquidambar orientalis, Lupinus spp, Lycopersicon esculentum, Macadamia spp, Macroptylium spp, Magnolia spp, Malva spp, Melaleuca spp, Melissa officinalis, Mentha spp, Micromeria spp, Morus alba, Myrtus communis, Nasturtium officinale, Nerium oleander, Nigella sativa, Ocimum spp, Oenanthe spp, Olea europaea, Ononis spinosa, Opuntia ficus-indica, Origanum spp, Paeonia spp, Panax spp, Panicum maximum, Passiflora spp, Phaseolus spp, Phyllanthus spp, Physalis spp, Pinus spp, Piper spp, Plantago spp, Podophyllum spp, Polygonum spp, Prunus spp, Punica granatum, Pycnanthemum spp, Quercus spp, Rosa spp, Rosmarinus officinalis, Rubus spp, Rumex spp, Ruscus aculeatus, Ruta graveolens, Salix alba, Salvia spp, Santalum album, Santolina chamaecyparissus, Satureja spp, Schinus molle, Scrophularia spp, Scutellaria spp, Senna spp, Serenoa repens, Sesamum indicum, Sideritis spp, Silybum marianum, Simmondsia chinensis, Smilax spp, Solanum spp, Spirulina spp., Stachys officinalis, Stevia rebaudiana, Styrax benzoin, Syzygium spp, Tagetes spp, Taraxacum officinale, Taxus spp, Thymus spp, Tilia spp, Trifolium spp, Trigonella foenum-graecum, Triticum aestivum, Ulex spp, Uncaria spp, Urtica dioica, Vaccinium spp, Valeriana officinalis, Vanilla planifolia, Verbascum Thapsus, Verbena officinalis, Viburnum opulus, Vinca spp, Viscum album, Vitex agnus-castus, Vitis vinifera, Yucca baccata, Zea mays, Zingiber officinale, and a combination thereof.

45. The composition according to claim 43, wherein the preservative is between 0.1 wt % and 3 wt % of the composition.

46. The composition according to claim 36, further comprising a film-forming biopolymer, wherein the biopolymer is between 0.1 wt % and 4 wt % of the composition.

47. The composition according to claim 46, wherein the biopolymer is selected from the group consisting of pullulan or other polysaccharides, galactomannans, Turkish tragacanth, locust bean gum or other gums, alginates, casein or caseinates, and a combination thereof.

48. A composition for transdermal delivery, the composition comprising:
an oily phase including an oil between 0.1 wt % and 6 wt % of the composition;
an aqueous phase including an herbal extract solution having soluble components of a subject plant solvated in an extraction medium,
wherein the herbal extract solution is one of an infusion, a decoction, a maceration, or a product from another extraction technique on the subject plant,
wherein the herbal extract solution is produced from an extraction technique on the subject plant using 1 weight unit of the subject plant to 6-12 weight units of the extraction medium,
wherein the extraction medium includes soluble components of a saponin plant solvated in water,
wherein the extraction medium is produced from an extraction technique on the saponin plant using 1 weight unit of the saponin plant to 5-10 weight units of water, wherein the extraction medium is one of an infusion, a decoction, a maceration, or a product from another extraction technique on the saponin plant,
wherein the saponin plant and the subject plant are different types of plants; and
a preservative providing antioxidant and/or antimicrobial properties, wherein the preservative is between 0.1 wt % and 3 wt % of the composition.

49. The composition according to claim 48, wherein the subject plant is selected from the group consisting of Achillea Millefolium, Aesculus Hippocastanum, Agrimonia Aupatoria, Alchemilla Vulgaris, Allium Sativum, Althaea Officinalis, Angelica Archangelica, Angelica sinensis syn. A. polymorpha, Anthemis Nobilis, Apium Graveolens, Arctostaphylos Uva Ursi, Arnica Montana, Artemisia Vulgaris, Articum Lappa, Aspalathus Linearis, Astragalus membranaceus, Berberis Vulgaris, Betula Alba, Borago Officinalis, Calendula Officincalis, Calluna Vulgaris, Camellia Sinensis, Capsella Bursa Pastoris, Capsicum Annuum, Cardus Benedictus, Cardus Marianus, Carum Carvi, Cassia Angustifolia, Centella asiatica syn. Hydrocotyle, Ceranus Vulgaris, Ceratonia siliqua, Chamaecrista mimosoides (L.), Cichorium Intybus, Cimicifuga racemosa, Cinnamomum Zeylanicum, Citrus paradisi Macfadyen, Citrus Aurantium, Citrus Limonum, Citrus Sinensis, Cnidium monnieri, Coffea Cruda, Cordyceps sinensis (Berk) Sacc., Cortex Magnoliae Officinalis, Corylus Avellana, Crataegus monogyna, Crataegus cuneata Sieb. et Zucc., Crataegus Oxyacantha, Cucurbita Pepo, Cupressus Sempervirens, Curcuma Longa, Cymbopogon Flexuosus, Cyminum Cuminum, Cynara Scolymus, Daucus Carota, Dioscoreavillosa, Echinacea Angustifolia, Echinacea Purpurea, Elettaria Cardamomum, Eleutherococcus Senticosus, Eleutherococcus senticosus, Acanthopanax senticosus, Epimedium brevicornum, Equisetum Arvense, Erigeron Canadensis, Eschscholtzia Californica, Eucalyptus Globulus, Ferula elaeochytris, Foeniculum Vulgare, Fragaria Vesca, Fraxinus Exelsior, Fructus Schisandrae, Fumaria Officinalis, Galla Chinensis, Gentiana Lutea, Ginkgo Biloba, Glechoma Hederaceum, Glycine max, Glycyrrhiza Glabra, Hamamelis Virginiana, Harpagophytum, Hibiscus Sabdariffa, Hieracium Pilosella, Humulus Lupulus, Hypericum Perforatum, Hysopus Officinalis, Juglans Regia, Juniperus Communis, Lavandula Angustifolia, Levisticum Officinalis, Ligustrum lucidum, Ait. or Eriobotrya japonica (Thunb.) Lindl. L., Lippia Citriodora, Lotus Corniculatus, Lycium Barbarum, Lycopersicum esculentum, Malva Sylvestris, Matricaria Chamomilla, Medicago Sativa, Melilotus Officinalis, Melissa Officinalis, Mentha Piperita, Mentha Viridis, Momordica Charantia, Ocimum Basilicium, Olea Europea, Ononis Spinosa, Origanum Majorana, Origanum Vulgaris, Paeonia Albiflora Root, Paeonia suffruticosa, Passiflora Incarnata, Passiflora quadrangularis, Paullinia Cupana, Perilla frutescens (L.) Britton., Phaseolus Vulgaris, Pimpinella Anisum, Pinus Pinaster, Pinus strobes, Pinus Sylvestris, Plantago Lanceolata, Plantago Major, Pneumus Boldus, Polygonum cuspidatum sieb. et zucc, Primula Officinalis, Pueraria lobata (Willd.) Ohwi, Pulmonaria Officinalis, Punica granatum L., Punica Granatum, Pyrus malus, Quercus Robur, Quercus spp., Raphanus Sativus, Rhamnus Frangula, Rheum Officinalis, Rhodiola rosea L., Ribers Nigrum, Rosa Canina, Rosmarinus Officinalis, Rubus fructicosus, Rubus Ideaus, Ruscus Aculeatus, Sabal Serrulata, Salix Alba, Salvia miltiorrhiza, Salvia Officinalis, Salvia Sclarea, Sambucus Nigra, Satureja Montana, Serenoa repens, Silybum marianum (L.) Gaertner, Soja Hispida, Solidago Virga Aurea, Spinacia Oleracea, Spirea Ulmaria, Symphytum officinalis, Taraxacum officinale, Taraxacum Officinalis, Thymus sp., Thymus vulgaris, Tilia Officinalis, Tribulus terrestris, Trigonella, Uncaria Tomentosa, Urtica Dioica, Valeriana Officinalis, Verbascum Thapsus, Verbena Officinalis, Viburnum Lanta, Viola Tricolor, Viscum Album, Vitex Agnus Castus, Vitis Vinifera, Zea Mais, Zingiber Officinalis, and a combination thereof.

50. The composition according to claim 48, wherein the saponin plant is selected from the group consisting of Camellia sinensis, Styrax japonica, Acacia concinna, Acacia nilotica, Acorus calamus, Aesculus hippocastanum, Agave Americana, Ailanthus altissima, Akebia quinata, Albizia julibrissin, Aletris farinose, Aleurites fordii, Allium cepa, Allium drummondii, Allium fistulosum, Allium neapolitanum, Allium oleraceum, Allium ramosum, Allium sativum var. sativum, Allium schoenoprasum, Allium sphaerocephalon, Allium tricoccum, Allium tuberosum, Allium ursinum, Allium vineale, Anemone pulsatilla, Arisaema consanguineum, Asparagus lucidus, Balanites aegyptiacus, Banisteriopsis caapi, Bupleurum falcatum, Calendula officinalis, Camellia oleifera, Capparis spinosa, Capsicum annum, Castanospermum austral, Caulophyllum thalictroides, Centella asiatica, Ceratonia siliqua, Chrysophyllum cainito, Cissampelos pareira, Coix lacryma-jobi, Colocasia esculenta, Colubrina arborescens, Corchorus olitorius, Cymbopogon citrates, Desmodium, Dianthus caryophyllus, Dictamnus albus, Dioscorea bulbifera, Dodonaea viscose, Elytrigia repens, Eryngium foetidum, Glechoma hederacea, Guaiacum sp., Gypsophila acantholimoides, Gypsophila Achaia, Gypsophila acutifolia, Gypsophila adenophora, Gypsophila adenophylla, Gypsophila albida, Gypsophila altissima, Gypsophila antari, Gypsophila antoninae, Gypsophila arabica, Gypsophila aretioides, Gypsophila arrostii, Gypsophila aucheri, Gypsophila aulieatensis, Gypsophila australis, Gypsophila bicolor, Gypsophila capituliflora, Gypsophila cephalotes, Gypsophila cerastioides, Gypsophila davurica, Gypsophila desertorum, Gypsophila elegans, Gypsophila fastigiata, Gypsophila glandulosa, Gypsophila glomerata, Gypsophila huashanensis, Gypsophila imbricate, Gypsophila intricate, Gypsophila iranica, Gypsophila krascheninnikovii, Gypsophila libanotica, Gypsophila licentiana, Gypsophila muralis, Gypsophila nana, Gypsophila oldhamiana, Gypsophila pacifica, Gypsophila paniculata, Gypsophila patrinii, Gypsophila perfoliata, Gypsophila petraea, Gypsophila pilosa, Gypsophila repens, Gypsophila rokejeka, Gypsophila ruscifolia, Gypsophila scorzonerifolia, Gypsophila sericea, Gypsophila silenoides, Gypsophila sp., Gypsophila spinosa, Gypsophila stevenii, Gypsophila struthium, Gypsophila tenuifolia, Gypsophila tschiliensis, Gypsophila uralensis, Gypsophila venusta, Gypsophila viscose, Gypsophila wendelboi, Gypsophila wilhelminae, Gypsophila xanthochlora, Hibiscus sabdariffa, Hyacinthus orientalis, Hyacinthus orientalis, Hydrangea arborescens, Hypericum perforatum, Lagenaria siceraria, Leonurus cardiac, Lonicera japonica, Manilkara zapota, Medicago sativa, Melia azedarach, Menyanthes trifoliate, Ocimum basilicum, Origanum majorana, Panax japonicas, Panax quinquefolius, Panax quinqufolius, Paullinia cupana, Phragmites australis, Polygala tenuifolia, Portulaca oleracea, Quillaja saponaria, Rosa centifolia, Rosa gallica, Rosa laevigata, Ruscus aculeatus, Salvia officinalis, Sapindus delavayi, Sapindus marginatus, Sapindus mukorossi, Sapindus saponaria, Sapindus trifoliatus, Saponaria officinalis, Sida rhombifolia, Smilax spp, Solanum lycopersicum, Solanum melongena, Solanum nigrum, Taraxacum officinale, Terminalia bellirica, Teucrium chamaedrys, Teucrium maxum, Teucrium montanum, Teucrium scordium, Tilia sp., Trigonella foenum-graecum, Vinca minor, Viola tricolor, Yucca schidigera, Zea mays, Zizyphus jujube, another plant including saponin, and a combination thereof.

51. The composition according to claim 48, wherein the target plant is selected from the group consisting of Pinus mugo, Rosa damascene, Abies alba, Abies siberica ledeb., Acorus calamus L., Agathosma betulina, Allium sativum L., Amyris balsamifera L., Anethum graveolens, Angelica archangelica L., Aniba rosaeodora Ducke, Anthemis nobilis, Apium graveolens, Artemisia dracunculus L, Artemisia herba-alba Asso, Artemisia pallens Wall., Boswellia carterii, Bulnesia sarmientoi, Cananga odorata, Canarium commune, Carum Carvi L., Cedrus atlantica, Cinnamomum Camphora Ness & Eberm, Cinnamomum cassia, Cinnamomum zeylanicum Blume, Cistus ladaniferus L, Citrus aurantifolia Swingle, Citrus Aurantium, Citrus aurantium amara, Citrus aurantium ssp. Bergamia, Citrus bergamia, Citrus dulcis, Citrus limon, L. Burman, Citrus paradise, Citrus reticulata Blanco, Citrus sinensis, Coleus Forskholii, Commiphora Myrrha, Copaifera officinalis, Coriandrum sativum L, Croton eluteria, Cryptocaryo massoy, Cuminum cyminum L, Cupressus funebris, Cupressus sempervirens L., Cymbopogon flexuosus, Cymbopogon winterianus Jowitt, Daucus Carota Sativa, Elettaria cardamomum L. Maton, Eucalyptus citriodora Hooker, Eucalyptus globulus Labille, Eucalytus radiate, Eugenia caryophyllus, Ferula asafoetida L., Ferula gumosa, Ex-ferula galbaniflua boiss. & buhse, Foeniculum vulgare Miller, Gaultheria procumbens, Helichrysum italicum, Hyssopus officinalis, Illicium verum, Juniperus communis, Juniperus oxycedrus spp, Juniperus virginiana/mexicana, Laurus nobilis, Lavandula angustifolia p. Miller, Lavandula hybrid, Levisticum officinale, Litsea cubeba, Litsea cubeba Pers., Matricaria chamomilla, Matricaria recutita, Melaleuca alternifolia, Melaleuca Leucadendron (L.), Melaleuca Viridiflora, Melissa officinalis, Mentha arvensis, Mentha cardiac, Mentha citrate, Mentha piperita, Mentha pulegium, Mentha spicata, Mentha viridis, Michelia alba, Myristica frangrans, Myrocarpus frondosus, Myrtus communis L., Ocimum basilicum L, Origanum majorana, Origanum vulgare, Pelargonium graveolens, Petroselinum sativum, Pimenta dioica, Pimenta racemosa, Pimpinella anisum, Pinus pumilio, Pinus spp., Pinus sylvestris, Piper nigrum, Pogostemon cablin, Ravensara aromatica, Sonnerat, Rosa centifolia, Rosmarinus officinalis, Salvia hispanica, Salvia officinalis, Salvia sclarea, Santalum album, Santalum austrocaledonicum Vl, Sassafras Albidum M, Satureja hortensis, Satureja Montana, Syzygium aromaticum, Tagetes glandullfera Schrank, Tanacetum Vulgare L., Thuya Occidentalis, Thymus mastichina L., Thymus Serpylum L., Thymus vulgaris, Thymus zygis, Traschispermum ammi, Vetyveria zizanioides, Zingiber officinalis, Prunus amygdalus var. dulcis, Persea gratissima, Prunus armeniaca, Borage officinalis, Camellia japonica, Cocos nucifera, Oenothera biennis, Vitis vinifera, Corylus avellana, Simmondsia chinensis, Aleurites moluccana, Macadamia tetraphylla, Oleo europaea, Rosa Rubiginosa, Sesamum indicum, Helianthus annuus, Tiriticum aestivum, and a combination thereof.

52. The composition according to claim 48, wherein the preservative is selected from the group consisting of Acacia ssp, Achillea millefolium, Allium cepa, Allium sativum var. sativum, Aloe spp., Aloe vera, Althaea officinalis, Anethum graveolens, Angelica spp, Apium graveolens, Arachis hypogaea, Artemisia spp, Astragalus membranaceus, Avena sativa, Azadirachta indica, Berberis vulgaris, Beta vulgaris, Borago officinalis, Brassica spp, Camellia sinensis, Capparis spinosa, Capsella bursa-pastoris, Capsicum annuum, Capsicum frutescens, Carum carvi, Castanea sativa, Centella asiatica, Ceratonia siliqua, Chrysanthemum spp, Cinchona spp, Cinnamomum ssp, Citrus ssp, Coffea Arabica, Coriandrum sativum, Crataegus spp, Cucurbita spp, Daucus carota, Digitalis purpurea, Echinacea angustifolia, Echinacea pallid, Elaeagnus angustifolia, Ephedra spp, Eruca sativa, Eucalyptus spp, Ferula spp, Fragaria spp, Garcinia spp, Genista spp, Gentiana lutea, Ginkgo biloba, Glycine max, Glycyrrhiza glabra, Gossypium spp, Hedera helix, Helianthus annuus, Hibiscus rosa-sinensis, Hordeum vulgare, Hydrangea spp, Hypericum perforatum, Inula helenium, Jasminum officinale, Juglans spp, Juniperus spp, Lactuca sativa, Laurus nobilis, Lavandula spp, Lippia spp, Liquidambar orientalis, Lupinus spp, Lycopersicon esculentum, Macadamia spp, Macroptylium spp, Magnolia spp, Malva spp, Melaleuca spp, Melissa officinalis, Mentha spp, Micromeria spp, Morus alba, Myrtus communis, Nasturtium officinale, Nerium oleander, Nigella sativa, Ocimum spp, Oenanthe spp, Olea europaea, Ononis spinosa, Opuntia ficus-indica, Origanum spp, Paeonia spp, Panax spp, Panicum maximum, Passiflora spp, Phaseolus spp, Phyllanthus spp, Physalis spp, Pinus spp, Piper spp, Plantago spp, Podophyllum spp, Polygonum spp, Prunus spp, Punica granatum, Pycnanthemum spp, Quercus spp, Rosa spp, Rosmarinus officinalis, Rubus spp, Rumex spp, Ruscus aculeatus, Ruta graveolens, Salix alba, Salvia spp, Santalum album, Santolina chamaecyparissus, Satureja spp, Schinus molle, Scrophularia spp, Scutellaria spp, Senna spp, Serenoa repens, Sesamum indicum, Sideritis spp, Silybum marianum, Simmondsia chinensis, Smilax spp, Solanum spp, Spirulina spp., Stachys officinalis, Stevia rebaudiana, Styrax benzoin, Syzygium spp, Tagetes spp, Taraxacum officinale, Taxus spp, Thymus spp, Tilia spp, Trifolium spp, Trigonella foenum-graecum, Triticum aestivum, Ulex spp, Uncaria spp, Urtica dioica, Vaccinium spp, Valeriana officinalis, Vanilla planifolia, Verbascum Thapsus, Verbena officinalis, Viburnum opulus, Vinca spp, Viscum album, Vitex agnus-castus, Vitis vinifera, Yucca baccata, Zea mays, Zingiber officinale, and a combination thereof.

53. The composition according to claim 48, further comprising a film-forming biopolymer between 0.1 wt % and 4 wt % of the composition, wherein the biopolymer is selected from the group consisting of pullulan or other polysaccharides, galactomannans, Turkish tragacanth, locust bean gum or other gums, alginates, casein or caseinates, and a combination thereof.

54. A method for treating human skin, the method comprising applying, to a skin surface, the composition according to claim 36.

55. A method for enhanced herbal extraction and transdermal delivery of an herbal extract solution, the method comprising:
extracting soluble components of a saponin plant with water to provide an extraction medium;
extracting soluble components of a subject plant with the extraction medium to provide an herbal extract solution; and
mixing the herbal extract solution with a component of complimentary function to produce a product for transdermal delivery.

56. The method according to claim 55, wherein the subject plant is selected from the group consisting of Achillea Millefolium, Aesculus Hippocastanum, Agrimonia Aupatoria, Alchemilla Vulgaris, Allium Sativum, Althaea Officinalis, Angelica Archangelica, Angelica sinensis syn. A. polymorpha, Anthemis Nobilis, Apium Graveolens, Arctostaphylos Uva Ursi, Arnica Montana, Artemisia Vulgaris, Articum Lappa, Aspalathus Linearis, Astragalus membranaceus, Berberis Vulgaris, Betula Alba, Borago Officinalis, Calendula Officincalis, Calluna Vulgaris, Camellia Sinensis, Capsella Bursa Pastoris, Capsicum Annuum, Cardus Benedictus, Cardus Marianus, Carum Carvi, Cassia Angustifolia, Centella asiatica syn. Hydrocotyle, Ceranus Vulgaris, Ceratonia siliqua, Chamaecrista mimosoides (L.), Cichorium Intybus, Cimicifuga racemosa, Cinnamomum Zeylanicum, Citrus paradisi Macfadyen, Citrus Aurantium, Citrus Limonum, Citrus Sinensis, Cnidium monnieri, Coffea Cruda, Cordyceps sinensis (Berk) Sacc., Cortex Magnoliae Officinalis, Corylus Avellana, Crataegus monogyna, Crataegus cuneata Sieb. et Zucc., Crataegus Oxyacantha, Cucurbita Pepo, Cupressus Sempervirens, Curcuma Longa, Cymbopogon Flexuosus, Cyminum Cuminum, Cynara Scolymus, Daucus Carota, Dioscoreavillosa, Echinacea Angustifolia, Echinacea Purpurea, Elettaria Cardamomum, Eleutherococcus Senticosus, Eleutherococcus senticosus, Acanthopanax senticosus, Epimedium brevicornum, Equisetum Arvense, Erigeron Canadensis, Eschscholtzia Californica, Eucalyptus Globulus, Ferula elaeochytris, Foeniculum Vulgare, Fragaria Vesca, Fraxinus Exelsior, Fructus Schisandrae, Fumaria Officinalis, Galla Chinensis, Gentiana Lutea, Ginkgo Biloba, Glechoma Hederaceum, Glycine max, Glycyrrhiza Glabra, Hamamelis Virginiana, Harpagophytum, Hibiscus Sabdariffa, Hieracium Pilosella, Humulus Lupulus, Hypericum Perforatum, Hysopus Officinalis, Juglans Regia, Juniperus Communis, Lavandula Angustifolia, Levisticum Officinalis, Ligustrum lucidum, Aitor Eriobotrya japonica (Thunb.) Lindl. L., Lippia Citriodora, Lotus Corniculatus, Lycium Barbarum, Lycopersicum esculentum, Malva Sylvestris, Matricaria Chamomilla, Medicago Sativa, Melilotus Officinalis, Melissa Officinalis, Mentha Piperita, Mentha Viridis, Momordica Charantia, Ocimum Basilicium, Olea Europea, Ononis Spinosa, Origanum Majorana, Origanum Vulgaris, Paeonia Albillora Root, Paeonia suffruticosa, Passiflora Incarnata, Passiflora quadrangularis, Paullinia Cupana, Perilla frutescens (L.) Britton., Phaseolus Vulgaris, Pimpinella Anisum, Pinus Pinaster, Pinus strobes, Pinus Sylvestris, Plantago Lanceolata, Plantago Major, Pneumus Boldus, Polygonum cuspidatum sieb. et zucc, Primula Officinalis, Pueraria lobata (Willd.) Ohwi, Pulmonaria Officinalis, Punica granatum L., Punica Granatum, Pyrus malus, Quercus Robur, Quercus spp., Raphanus Sativus, Rhamnus Frangula, Rheum Officinalis, Rhodiola rosea L., Ribers Nigrum, Rosa Canina, Rosmarinus Officinalis, Rubus fructicosus, Rubus Ideaus, Ruscus Aculeatus, Sabal Serrulata, Salix Alba, Salvia miltiorrhiza, Salvia Officinalis, Salvia Sclarea, Sambucus Nigra, Satureja Montana, Serenoa repens, Silybum marianum (L.) Gaertner, Soja Hispida, Solidago Virga Aurea, Spinacia Oleracea, Spirea Ulmaria, Symphytum officinalis, Taraxacum officinale, Taraxacum Officinalis, Thymus sp., Thymus vulgaris, Tilia Officinalis, Tribulus terrestris, Trigonella, Uncaria Tomentosa, Urtica Dioica, Valeriana Officinalis, Verbascum Thapsus, Verbena Officinalis, Viburnum Lanta, Viola Tricolor, Viscum Album, Vitex Agnus Castus, Vitis Vinifera, Zea Mais, Zingiber Officinalis, and a combination thereof.

57. The method according to claim 55, wherein the saponin plant is selected from the group consisting of Camellia sinensis, Styrax japonica, Acacia concinna, Acacia nilotica, Acorus calamus, Aesculus hippocastanum, Agave Americana, Ailanthus altissima, Akebia quinata, Albizia julibrissin, Aletris farinose, Aleurites fordii, Allium cepa, Allium drummondii, Allium fistulosum, Allium neapolitanum, Allium oleraceum, Allium ramosum, Allium sativum var. sativum, Allium schoenoprasum, Allium sphaerocephalon, Allium tricoccum, Allium tuberosum, Allium ursinum, Allium vineale, Anemone pulsatilla, Arisaema consanguineum, Asparagus lucidus, Balanites aegyptiacus, Banisteriopsis caapi, Bupleurum falcatum, Calendula officinalis, Camellia oleifera, Capparis spinosa, Capsicum annuum, Castanospermum austral, Caulophyllum thalictroides, Centella asiatica, Ceratonia siliqua, Chrysophyllum cainito, Cissampelos pareira, Coix lacryma-jobi, Colocasia esculenta, Colubrina arborescens, Corchorus olitorius, Cymbopogon citrates, Desmodium, Dianthus caryophyllus, Dictamnus albus, Dioscorea bulbifera, Dodonaea viscose, Elytrigia repens, Eryngium foetidum, Glechoma hederacea, Guaiacum sp., Gypsophila acantholimoides, Gypsophila Achaia, Gypsophila acutifolia, Gypsophila adenophora, Gypsophila adenophylla, Gypsophila albida, Gypsophila altissima, Gypsophila antari, Gypsophila antoninae, Gypsophila arabica, Gypsophila aretioides, Gypsophila arrostii, Gypsophila aucheri, Gypsophila aulieatensis, Gypsophila australis, Gypsophila bicolor, Gypsophila capituliflora, Gypsophila cephalotes, Gypsophila cerastioides, Gypsophila davurica, Gypsophila desertorum, Gypsophila elegans, Gypsophila fastigiata, Gypsophila glandulosa, Gypsophila glomerata, Gypsophila huashanensis, Gypsophila imbricate, Gypsophila intricate, Gypsophila iranica, Gypsophila krascheninnikovii, Gypsophila libanotica, Gypsophila licentiana, Gypsophila muralis, Gypsophila nana, Gypsophila oldhamiana, Gypsophila pacifica, Gypsophila paniculata, Gypsophila patrinii, Gypsophila perfoliata, Gypsophila petraea, Gypsophila pilosa, Gypsophila repens, Gypsophila rokejeka, Gypsophila ruscifolia, Gypsophila scorzonerifolia, Gypsophila sericea, Gypsophila silenoides, Gypsophila sp., Gypsophila spinosa, Gypsophila stevenii, Gypsophila struthium, Gypsophila tenuifolia, Gypsophila tschiliensis, Gypsophila uralensis, Gypsophila venusta, Gypsophila viscose, Gypsophila wendelboi, Gypsophila wilhelminae, Gypsophila xanthochlora, Hibiscus sabdariffa, Hyacinthus orientalis, Hyacinthus orientalis, Hydrangea arborescens, Hypericum perforatum, Lagenaria siceraria, Leonurus cardiac, Lonicera japonica, Manilkara zapota, Medicago sativa, Melia azedarach, Menyanthes trifoliate, Ocimum basilicum, Origanum majorana, Panax japonicas, Panax quinquefolius, Panax quinqufolius, Paullinia cupana, Phragmites australis, Polygala tenuifolia, Portulaca oleracea, Quillaja saponaria, Rosa centifolia, Rosa gallica, Rosa laevigata, Ruscus aculeatus, Salvia officinalis, Sapindus delavayi, Sapindus marginatus, Sapindus mukorossi, Sapindus saponaria, Sapindus trifoliatus, Saponaria officinalis, Sida rhombifolia, Smilax spp, Solanum lycopersicum, Solanum melongena, Solanum nigrum, Taraxacum officinals, Terminalia bellirica, Teucrium chamaedrys, Teucrium maxum, Teucrium montanum, Teucrium scordium, Tilia sp., Trigonella foenum-graecum, Vinca minor, Viola tricolor, Yucca schidigera, Zea mays, Zizyphus jujube, another plant including saponin, and a combination thereof.

58. The method according to claim 55, wherein the extracting of soluble components of a saponin plant with water includes using 1 weight unit of the saponin plant to 5-10 weight units of water.

59. The method according to claim 55, wherein the component of complimentary function is selected from the group consisting of film-forming biopolymers, rheology modifiers, anti-pollution ingredients, UV screeners, hyaluronic acid, ceramides, and humectants including vinegar or betaine.

60. The method according to claim 55, wherein the component of complimentary function is an oil to form an emulsion.

61. The method according to claim 60, wherein the oil is a fixed oil and/or an essential oil of a target plant, the target plant being selected from the group consisting of Pinus mugo, Rosa damascene, Abies alba, Abies siberica ledeb., Acorus calamus L., Agathosma betulina, Allium sativum L., Amyris balsamifera L., Anethum graveolens, Angelica archangelica L., Aniba rosaeodora Ducke, Anthemis nobilis, Apium graveolens, Artemisia dracunculus L, Artemisia herba-alba Asso, Artemisia pallens Wall., Boswellia carterii, Bulnesia sarmientoi, Cananga odorata, Canarium commune, Carum Carvi L., Cedrus atlantica, Cinnamomum Camphora Ness & Eberm, Cinnamomum cassia, Cinnamomum zeylanicum Blume, Cistus ladaniferus L, Citrus aurantifolia Swingle, Citrus Aurantium, Citrus aurantium amara, Citrus aurantium ssp. Bergamia, Citrus bergamia, Citrus dulcis, Citrus limon, L. Burman, Citrus paradise, Citrus reticulata Blanco, Citrus sinensis, Coleus Forskholii, Commiphora Myrrha, Copaifera officinalis, Coriandrum sativum L, Croton eluteria, Cryptocaryo massoy, Cuminum cyminum L, Cupressus funebris, Cupressus sempervirens L., Cymbopogon flexuosus, Cymbopogon winterianus Jowitt, Daucus Carota Sativa, Elettaria cardamomum L. Maton, Eucalyptus citriodora Hooker, Eucalyptus globulus Labille, Eucalytus radiate, Eugenia caryophyllus, Ferula asafoetida L., Ferula gumosa, Ex-ferula galbaniflua boiss. & buhse, Foeniculum vulgare Miller, Gaultheria procumbens, Helichrysum italicum, Hyssopus officinalis, Illicium verum, Juniperus communis, Juniperus oxycedrus spp, Juniperus virginiana/mexicana, Laurus nobilis, Lavandula angustifolia p. Miller, Lavandula hybrid, Levisticum officinale, Litsea cubeba, Litsea cubeba Pers., Matricaria chamomilla, Matricaria recutita, Melaleuca alternifolia, Melaleuca Leucadendron (L.), Melaleuca Viridiflora, Melissa officinalis, Mentha arvensis, Mentha cardiac, Mentha citrate, Mentha piperita, Mentha pulegium, Mentha spicata, Mentha viridis, Michelia alba, Myristica frangrans, Myrocarpus frondosus, Myrtus communis L., Ocimum basilicum L, Origanum majorana, Origanum vulgare, Pelargonium graveolens, Petroselinum sativum, Pimenta dioica, Pimenta racemosa, Pimpinella anisum, Pinus pumilio, Pinus spp., Pinus sylvestris, Piper nigrum, Pogostemon cablin, Ravensara aromatica, Sonnerat, Rosa centifolia, Rosmarinus officinalis, Salvia hispanica, Salvia officinalis, Salvia sclarea, Santalum album, Santalum austrocaledonicum Vl, Sassafras Albidum M, Satureja hortensis, Satureja Montana, Syzygium aromaticum, Tageles glandullfera Schrank, Tanacetum Vulgare L., Thuya Occidentalis, Thymus mastichina L., Thymus Serpylum L., Thymus vulgaris, Thymus zygis, Traschispermum ammi, Vetyveria zizanioides, Zingiber officinalis, Prunus amygdalus var. dulcis, Persea gratissima, Prunus armeniaca, Borage officinalis, Camellia japonica, Cocos nucifera, Oenothera biennis, Vitis vinifera, Corylus avellana, Simmondsia chinensis, Aleurites moluccana, Macadamia tetraphylla, Oleo europaea, Rosa Rubiginosa, Sesamum indicum, Helianthus annuus, Tiriticum aestivum, and a combination thereof.

62. The method according to claim 55, wherein the component of complimentary function is a preservative selected from the group consisting of Acacia ssp, Achillea millefolium, Allium cepa, Allium sativum var. sativum, Aloe spp., Aloe vera, Althaea officinalis, Anethum graveolens, Angelica spp, Apium graveolens, Arachis hypogaea, Artemisia spp, Astragalus membranaceus, Avena sativa, Azadirachta indica, Berberis vulgaris, Beta vulgaris, Borago officinalis, Brassica spp, Camellia sinensis, Capparis spinosa, Capsella bursa-pastoris, Capsicum annuum, Capsicum frutescens, Carum carvi, Castanea sativa, Centella asiatica, Ceratonia siliqua, Chrysanthemum spp, Cinchona spp, Cinnamomum ssp, Citrus ssp, Coffea Arabica, Coriandrum sativum, Crataegus spp, Cucurbita spp, Daucus carota, Digitalis purpurea, Echinacea angustifolia, Echinacea pallid, Elaeagnus angustifolia, Ephedra spp, Eruca sativa, Eucalyptus spp, Ferula spp, Fragaria spp, Garcinia spp, Genista spp, Gentiana lutea, Ginkgo biloba, Glycine max, Glycyrrhiza glabra, Gossypium spp, Hedera helix, Helianthus annuus, Hibiscus rosa-sinensis, Hordeum vulgare, Hydrangea spp, Hypericum perforatum, Inula helenium, Jasminum officinale, Juglans spp, Juniperus spp, Lactuca sativa, Laurus nobilis, Lavandula spp, Lippia spp, Liquidambar orientalis, Lupinus spp, Lycopersicon esculentum, Macadamia spp, Macroptylium spp, Magnolia spp, Malva spp, Melaleuca spp, Melissa officinalis, Mentha spp, Micromeria spp, Morus alba, Myrtus communis, Nasturtium officinale, Nerium oleander, Nigella sativa, Ocimum spp, Oenanthe spp, Olea europaea, Ononis spinosa, Opuntia ficus-indica, Origanum spp, Paeonia spp, Panax spp, Panicum maximum, Passiflora spp, Phaseolus spp, Phyllanthus spp, Physalis spp, Pinus spp, Piper spp, Plantago spp, Podophyllum spp, Polygonum spp, Prunus spp, Punica granatum, Pycnanthemum spp, Quercus spp, Rosa spp, Rosmarinus officinalis, Rubus spp, Rumex spp, Ruscus aculeatus, Ruta graveolens, Salix alba, Salvia spp, Santalum album, Santolina chamaecyparissus, Satureja spp, Schinus molle, Scrophularia spp, Scutellaria spp, Senna spp, Serenoa repens, Sesamum indicum, Sideritis spp, Silybum marianum, Simmondsia chinensis, Smilax spp, Solanum spp, Spirulina spp., Stachys officinalis, Stevia rebaudiana, Styrax benzoin, Syzygium spp, Tagetes spp, Taraxacum officinale, Taxus spp, Thymus spp, Tilia spp, Trifolium spp, Trigonella foenum-graecum, Triticum aestivum, Ulex spp, Uncaria spp, Urtica dioica, Vaccinium spp, Valeriana officinalis, Vanilla planifolia, Verbascum Thapsus, Verbena officinalis, Viburnum opulus, Vinca spp, Viscum album, Vitex agnus-castus, Vitis vinifera, Yucca baccata, Zea mays, Zingiber officinale, and a combination thereof.

63. The method according to claim 55, wherein the component of complimentary function is between 0.1 wt % and 3 wt % of the composition.

64. The method according to claim 55, further comprising processing a mixture of the subject plant and the extraction medium prior to the step of extracting soluble components of the subject plant, wherein processing the mixture of the subject plant and the extraction medium includes one of mixing, heating, cavitation, application of direct electric current, ultrasound mixing, and a combination thereof.

65. The method according to claim 55, further comprising processing a mixture of the saponin plant and water prior to the step of extracting soluble components of the saponin plant, wherein processing the mixture of the saponin plant and water includes one of mixing, heating, cavitation, application of direct electric current, ultrasound mixing, and a combination thereof.
